(19) United States
(12) Patent Application Publication
(10) Pub. No.: US 2021/0310014 A1
(43) Pub. Date: Oct. 7, 2021

(54) CONSTRUCTION OF PLANT RECOMBINANT GENE EXPRESSION CONTROL PLATFORM

(71) Applicant: KAZUSA DNA RESEARCH INSTITUTE, CHIBA (JP)

(72) Inventors: Hiroshi MASUMOTO, CHIBA (JP); Daisuke SHIBATA, CHIBA (JP); Jun-ichirou OHZEKI, CHIBA (JP); Koei OKAZAKI, CHIBA (JP); Kazuto KUGOU, CHIBA (JP); Koichiro OTAKE, CHIBA (JP); Jekson ROBERTLEE, CHIBA (JP)

(73) Assignee: KAZUSA DNA RESEARCH INSTITUTE, CHIBA (JP)

(21) Appl. No.: 17/266,378

(22) PCT Filed: Aug. 5, 2019
(86) PCT No.: PCT/JP2019/030783

§ 371 Date: Jun. 25, 2021

(30) Foreign Application Priority Data
Aug. 10, 2018 (JP) 2018-152008

(51) Int. Cl. C12N 15/82

(52) U.S. Cl. CPC C12N 15/8205

(57) ABSTRACT

Gene expression is regulated (ON/OFF switching) by regulating the chromatin structure, and a several tens of genes (multigene) set introduced into genetically modified crops is stably expressed by the regulation.

A plant recombinant gene expression regulatory platform DNA sequence comprising (1) an artificial alphoid DNA sequence having a hierarchical repetitive structure of an alphoid DNA that forms a centromere of a human chromosome and having a nucleotide sequence a part of which is replaced by a binding site of a gene expression regulator (inducer); and (2) a multigene (a plurality of genes) expression cassette sequence, wherein the artificial alphoid DNA sequence is linked to upstream (5′ side) and downstream (3′ side) of the cassette sequence; and a method for regulating the expression of a recombinant gene in a plant body using the sequence.

### SEQUENCE LISTING

A sequence listing in electronic (ASCII text file) format is filed with this application and incorporated herein by reference. The name of the ASCII text file is “2021_0055A_ST25.txt”; the file was created on Jun. 17, 2021; the size of the file is 85 KB.

### BACKGROUND OF THE INVENTION

### 1. Field of the Invention

The present invention relates to a plant recombinant gene expression regulatory or control (gene expression ON/OFF switching) platform DNA sequence, a plant gene recombinant vector set including the platform DNA sequence and so on, a genetically modified plant body in which the platform DNA sequence and so on are introduced into a chromosome (in the present specification, a “plant body” includes a part of the plant body, such as plant cells, tissue, and calli, or a part derived therefrom), and a method for regulating the expression of a recombinant gene in the plant body.

### 2. Description of the Related Art

As serious problems in putting genetically modified crops to practical use, it is known that a phenomenon in which high expression of a transgene causes growth inhibition and a phenomenon in which expression of an introduced gene becomes unstable with progress of generation are often observed (Stam, M., Mol, J. N. M., and Kooter, J. M., The Silence of Genes in Transgenic Plants (Review), Annals of Botany (1997), 79, 3-12) (FIG. 1). In commercialization of a genetically modified plant, it is necessary to produce a large number of genetically modified strains and examine them for a long period and to select a stable strain. Accordingly, the main problem is that it takes time and an enormous development cost is required. The destabilization of recombinant gene expression is inferred to be caused by an epigenetic modification change in the chromatin structure at the gene introduction site (Stam, et al., 1997), but a method for preventing the destabilization has not been established yet.

The present inventors have succeeded in human and mouse cells to induce open-chromatinization (formation of openchromatin) capable of stably expressing a gene by directly binding a histone acetyltransferase and a histone chaperone to an ectopic chromosome insertion site of heterochromatinized synthetic repetitive DNA as a method for regulating the heterochromatinization (formation of heterochromatin) phenomenon (Non-Patent Literatures 1 to 4) (FIG. 2). This method for regulating the heterochromatin of an ectopic site using synthetic repetitive DNA has been developed by the present inventor through studies for producing a human artificial chromosome from repetitive DNA introduced into a cell (Non-Patent Literatures 5 and 6).

On the other hand, in plants, there has been an example of inducing expression of a plant gene by binding a fusion protein of a tetracycline repressor and a transcriptional activator to a tetracycline operator sequence introduced into the TATA-box upstream of the gene, so far (Non-Patent Literature 7).

However, although it has been succeeded in cells of mammals such as humans and mice to induce open chromatinization capable of stably expressing a gene as described above, in plants, no example has been reported on that expression of a gene is regulated by changing the chromatin structure through such histone modification.

Furthermore, it is known that in animal cells and plant cells, the structures and functions relating to the change in chromatin structure and gene expression regulation are different in many points. For example, DNA methylation modification is also closely involved in them, and in plant cells, methylation is introduced onto CHG and CHH (H is any of A, T, and C nucleotides) sequences in addition to methylation of a CG sequence which is observed in animal cells. Thus, plant cells have a more complicated regulation mechanism. In addition, histone H3 lysine 9 trimethylation (H3K9me3) forms heterochromatin in animals, but in plants, it is frequently detected also in the gene region to be transcribed (Non-Patent Literatures 8 and 9).

Accordingly, it cannot be predicted at all whether the mechanism of regulating the expression of a gene by a change in chromatin structure through histone modification, which is observed in mammalian cells, functions (exists) in the same way in plant body (cells) or not.

In addition, in existing genetically modified crops, emphasis is placed on the individual recombination of single or several genes, and a method for individually (singly) regulating the transcription of individual foreign genes introduced as described above by various transcriptional regulatory elements, expression regulators, and so on has been known. However, considering the rapid global expansion of synthetic biology, the demand for a new technology for gene introduction of a set of several tens of genes, such as gene introduction of the entire metabolic pathway, and for simultaneously, collectively, and stably expressing many genes is increasing.

### CITATION LIST

### Non-Patent Literature

Non-Patent Literature 1: Nakano, M., Cardinale, S., Noskov, V. N., Gassmann, R., Vagnarelli, P., Kandels-Lewis, S., Larionov, V., Earnshaw, W. C., and Masumoto, H., Inactivation of a human kinetochore by specific targeting of chromatin modifiers, Dev. Cell., 2008, Apr. 14 (4), 507-22.
Non-Patent Literature 2: Ohzeki, J., Bergmann, J. H., Kouprina, N., Noskov, V. N., Nakano, M., Kimura, H., Earnshaw, W. C., Larionov, V., and Masumoto, H., Breaking the HAC Barrier: histone H3K9 acetyl/methyl balance regulates CENP-A assembly, EMBO J., 2012, May 16, 31 (10), 2391-402.
Non-Patent Literature 3: Shono, N., Ohzeki, J., Otake, K., Martins, N. M. C., Nagase, T., Kimura, H., Larionov, V., Earnshaw, W. C., and Masumoto, H., Journal of Cell Science (2015) 128, 4572-4587, CENP-C and CENP-I are key connecting factors for kinetochore and CENP-A assembly.
Non-Patent Literature 4: Ohzeki, J., Shono, N., Otake, K., Martins, N. M., Kugou, K., Kimura, H., Nagase, T., Larionov, V., Earnshaw, W. C., and Masumoto, H., KAT7/HBO1/MYST2 Regulates CENP-A Chromatin Assembly by Antagonizing Suv39h1-Mediated Centromere Inactivation, Dev. Cell., 2016, June 6, 37 (5), 413-27.
Non-Patent Literature 5: Ikeno, M., Grimes, B., Okazaki, T., Nakano, M., Saitoh, K., Hoshino, H., McGill, N. I., Cooke, H., and Masumoto, H., Nature Biotechnology, 1998, 16, 431-439, Construction of YAC-based mammalian artificial chromosomes.
Non-Patent Literature 6: Okada, T., Ohzeki. J, Nakano, M., Yoda, M., Brinkley, W. R., Larionov, V., and Masumoto, H., Cell, 2007, 131, 1287-1300, CENP-B Controls Centromere Formation Depending on the Chromatin Context.
Non-Patent Literature 7: Weinmann, P., Gossen, M., Hillen, W., Bujard, H. and Gatz, C., Plant Journal, 1994, 5 (4), 559-569, A chimeric transactivator allows tetracycline-responsive gene expression in whole plant.
Non-Patent Literature 8: Jacob, Y., Feng, S., LeBlanc, C. A., Bernatavichute, Y. V., Stroud, H., Cokus, S., Johnson, L. M., Pellegrini, M., Jacobsen, S. E., and Michaels. S. D., Nature Structural & Molecular Biology, 2009, 16 (7), 763-769, ATXR5 and ATXR6 are H3K27 monomethyltransferases required for chromatin structure and gene silencing.
Non-Patent Literature 9: Roudier, F., Ahmed, I., Berard, C., Sarazin, A., Mary-Huard, T., Cortijo, S., Bouyer, D., Caillieux, E., Duvernois-Berthet, E., Al-Shikhley, L., Giraut, L., Despres, B., Drevensek, S., Barneche, F., Derozier, S., Brunaud, V., Aubourg, S., Schnittger, A., Bowler, C., Martin-Magniette, M. L., Robin, S., Caboche, M., and Colot, V. EMBO J., 2011, 30 (10), 1928-1938, Integrative epigenomic mapping defines four main chromatin states in Arabidopsis.

### SUMMARY OF THE INVENTION

Accordingly, it is a main purpose of the present invention to regulate gene expression (ON/OFF switching) by regulating the chromatin structure and to stably express a set of several tens of genes (multigene) introduced by the regulation in genetically modified crops (FIG. 3).

The present inventors constructed a gene expression ON/OFF switching platform composed of a combination of synthetic repetitive DNA having a centromere or heterochromatin-inducing function and open chromatinization induction by a histone modification enzyme and a histone chaperone for switching on/off the gene expression of a specific chromosome region. Furthermore, by introducing a site specific recombinant site into the platform as a base and selecting an animal or plant genomic DNA fragment (100 kbp or less) suitable for introducing several tens of genes, the inventors developed a construct that includes an expression ON/OFF switch and a recombinant site into which a foreign gene can be inserted freely and that can be introduced into a plant via Agrobacterium.

As a specific example, a DNA sequence (tetO) that can bind a histone modification enzyme-fused tetR protein was linked to a synthetic repetitive (repeating) DNA designed based on a human-derived centromere sequence as a factor necessary for heterochromatinization and induction of open chromatin, and the linked product was introduced together with a reporter gene into a tobacco cultured cell BY-2 and Arabidopsis thaliana and benthamiana tobacco plant individuals. Subsequently, histone modification enzyme-fused tetR (or rtetR) protein was expressed, and structural transformation of chromatin and expression of the reporter gene at the ectopic site were detected with a confocal laser fluorescence microscope to verify that gene expression can be regulated by the gene expression ON/OFF switching platform (FIG. 4).

The present invention has been accomplished based on the above unexpected new knowledge.

That is, the present invention includes the following aspects:

[Aspect 1] A plant recombinant gene expression regulatory platform DNA sequence comprising:

(1) an artificial alphoid DNA sequence having a hierarchical repetitive structure of an alphoid DNA that forms a centromere of a human chromosome and having a nucleotide sequence a part of which is replaced by a binding site of a gene expression regulator (inducer); and

(2) a multigene (a plurality of genes) expression cassette sequence, wherein

the artificial alphoid DNA sequence is linked to upstream (5′ side) and downstream (3′ side) of the cassette sequence;

[Aspect 2] The platform DNA sequence according to aspect 1, wherein the alphoid DNA is an α21-1 sequence of human chromosome 21;
[Aspect 3] The platform DNA sequence according to aspect 1 or 2, wherein the artificial alphoid DNA sequence has a 4 to 16-time repeated structure of a higher ordered repeat unit consisting of 11-mer of a repeat unit of 171 bp, and has a length of 7.5 to 30 kb;
[Aspect 4] The platform DNA sequence according to any one of aspects 1 to 3, comprising a nucleotide sequence including a binding site of the gene expression regulator in at least a part of the repeat units not containing a CENP-B box sequence;
[Aspect 5] The platform DNA sequence according to any one of aspects 1 to 4, wherein

the higher ordered repeat unit consisting of the 11-mer of the repeat unit constituting the artificial alphoid DNA sequence is a following DNA sequence:

a DNA sequence (1) consisting of the nucleotide sequence of SEQ ID NO: 1 (including tetO) or SEQ ID NO: 2 (including LacO);

a DNA sequence (2) that hybridizes with a DNA sequence consisting of a nucleotide sequence complementary to the nucleotide sequence of (1) under stringent conditions, the DNA sequence (2) consisting of a nucleotide sequence that includes a binding site of the gene expression regulator (inducer), and having substantially the same plant recombinant gene expression regulatory function as that of the DNA sequence (1); or

a DNA sequence (3) that consists of a nucleotide sequence showing a sequence identity of 80% or more to the whole of the nucleotide sequence of (1) and including a binding site of the gene expression regulator (inducer), the DNA sequence (3) having substantially the same plant recombinant gene expression regulatory function as that of the DNA sequence (1);

[Aspect 6] The platform DNA sequence according to any one of aspects 1 to 5, wherein the binding site of the gene expression regulator is tetO or LacO;
[Aspect 7] The platform DNA sequence according to any one of aspects 1 to 6, wherein the cassette sequence includes a recombinant enzyme site;
[Aspect 8] The platform DNA sequence according to any one of aspects 1 to 7, wherein the cassette sequence includes a marker gene or a reporter gene;
[Aspect 9] The platform DNA sequence according to any one of aspects 1 to 8, comprising several to ten-and-several genes or a multigene having a length of 5 to 100 kb introduced into the cassette sequence;
[Aspect 10] A plant recombinant gene expression regulatory platform vector comprising the platform DNA sequence according to aspects 1 to 9;
[Aspect 11] A DNA sequence for expressing the gene expression regulator according to aspect 1;
[Aspect 12] A gene expression regulator vector comprising the DNA sequence for expressing according to aspect 11;
[Aspect 13] The gene expression regulator vector according to aspect 12, wherein the artificial alphoid DNA sequence according to aspect 1 is linked to upstream (5′ side) and downstream (3′ side) of the DNA sequence for expression;
[Aspect 14] A plant gene recombinant fusion vector comprising the platform DNA sequence according to any one of aspects 1 to 9 and the DNA sequence for expressing according to aspect 11;
[Aspect 15] A plant gene recombinant vector set consisting of the plant recombinant gene expression regulatory platform vector according to aspect 9 and the gene expression regulator vector according to aspect 12 or 13;
[Aspect 16] A genetically modified plant body comprising a chromosome into which the plant recombinant gene expression regulatory platform DNA sequence according to any one of aspects 1 to 9 and the DNA sequence for expressing of the gene expression regulator according to aspect 11 have been introduced;
[Aspect 17] A method for manufacturing the plant body according to aspect 15, comprising introducing the fusion vector according to aspect 14 or the plant gene recombinant vector set according to aspect 15 into a plant body;
[Aspect 18] The manufacturing method according to aspect 17, using an Agrobacterium method;
[Aspect 19] A method for regulating the expression of a recombinant gene in the plant body according to aspect 15, the method comprising:

repressing or promoting the binding of the gene expression regulator to a binding site included in the artificial alphoid DNA sequence according to aspect 1 to regulate action of the gene expression regulator and to thereby transform a chromatin structure on the artificial alphoid DNA sequence and the multigene (a plurality of genes) expression cassette sequence between the heterochromatin and the open chromatin;

[Aspect 20] The regulating method according to aspect 19, wherein the repression or promotion of binding of the gene expression regulator to the binding site included in the artificial alphoid DNA sequence is performed by action of tetracycline (IPTG and the like); and
[Aspect 21] The regulating method according to aspect 19 or 20, wherein the transformation of the chromatin structure is induced by acetylation, methylation, and deacetylation of histone that are actions of the gene expression regulator.

The plant recombinant gene expression regulatory platform DNA sequence of the present invention regulates the action (activity) of the gene expression regulator (inducer) by promoting or repressing binding of the gene expression regulator (inducer) to a binding site (such as tetO) in the artificial alphoid DNA sequence regardless of the chromatin structure of the genome site into which the platform DNA sequence has been inserted. As a result, the chromatin structure on the artificial alphoid DNA sequence and the multigene (a plurality of genes) expression cassette sequence changes (transformation between open chromatin and heterochromatin), and expression regulation (gene expression ON/OFF switching) of a plurality of genes included in the multigene expression cassette sequence, which is a gene region sandwiched by the artificial alphoid DNA sequence, can be substantially simultaneously (cooperatively) performed.

Furthermore, in the present invention, the chromatin structure in the plant recombinant gene expression regulatory platform DNA sequence containing a transgene is artificially (actively) manipulated to regulate the expression of the introduced recombinant gene. Accordingly, the destabilization in recombinant gene expression that is caused by an epigenetic modification change in the chromatin structure can be prevented for several generations.

Incidentally, in the gene introduction using the gene expression ON/OFF switching platform of the present invention, the effect of reducing the number of copies per cell was observed. As a result, since a one-copy strain can be obtained with high probability using the technology of the present invention, the industrial usefulness of the invention is high.

### BRIEF DESCRIPTION OF THE DRAWINGS

FIG. 1 shows serious problems in putting genetically modified crops to practical use.

FIG. 2 shows gene expression regulation by chromatin structure transformation.

FIG. 3 shows problems in gene recombinant technology in plants.

FIG. 4 shows an example of the gene expression ON/OFF switching platform of the present invention.

FIG. 5 shows the structure (TAC vector #1) of an expression ON/OFF switching platform.

FIG. 6 shows a principle (I) of the expression ON/OFF switching platform.

FIG. 7 shows a principle (I): expression repression (OFF) of the expression ON/OFF switching platform.

FIG. 8 shows the principle (I): expression induction (ON) of the expression ON/OFF switching platform.

FIG. 9 shows a principle (II): self-activating type of the expression ON/OFF switching platform.

FIG. 10 shows a principle (II): self-activating type of the expression ON/OFF switching platform.

FIG. 11 shows structures and elongation synthesis of tetO, LacO, and alphoid DNAs.

FIG. 12 shows photographs of electrophoresis indicating the structures and elongation synthesis of tetO, LacO, and alphoid DNAs.

FIG. 13 shows production of Agrobacterium binary vector pRIBAC.

FIG. 14 shows production of Agrobacterium binary vector pRIBAC tetPF precursor plasmid.

FIG. 15 shows production of Agrobacterium binary vector pRIBAC tetPF precursor plasmid.

FIG. 16 shows production of Agrobacterium binary vector pRIBAC tetPF.

FIG. 17 shows a photograph of electrophoresis indicating the production of Agrobacterium binary vector pRIBAC tetPF.

FIG. 18 shows a photograph of electrophoresis indicating the production of Agrobacterium binary vector pRIBAC tetPF.

FIG. 19 shows production of Agrobacterium binary vector pRIBAC LacPF.

FIG. 20 shows a photograph of electrophoresis indicating the production of Agrobacterium binary vector pRIBAC LacPF.

FIG. 21 shows a photograph of electrophoresis indicating the production of Agrobacterium binary vector pRIBAC LacPF.

FIG. 22 shows production of Agrobacterium binary vector pRIBAC LacSG precursor plasmid (self-activating type).

FIG. 23 shows production of Agrobacterium binary vector pRIBAC LacSG precursor plasmid (self-activating type).

FIG. 24 shows production of Agrobacterium binary vector pRIBAC LacSG precursor plasmid (self-activating type).

FIG. 25 shows production of Agrobacterium binary vector pRIBAC LacSG plasmid (self-activating type).

FIG. 26 shows a photograph of electrophoresis indicating of production of Agrobacterium binary vector pRIBAC LacSG plasmid (self-activating type).

FIG. 27 shows photographs (EYFP fluorescence observation) indicating gene expression in expression ON/OFF switching platform introduced tobacco BY-2 strain.

FIG. 28 shows photographs (EYFP fluorescence observation/high magnification) indicating gene expression in expression ON/OFF switching platform introduced strain.

FIG. 29 shows photographs indicating gene expression (GUS staining) in expression ON/OFF switching platform introduced BY-2 strain.

FIG. 30 shows analysis by qPCR of the number of copies of introduced DNA in the expression ON/OFF switching platform introduced BY-2 strain.

FIG. 31 shows analysis of chromatin structure by ChIP of expression ON/OFF switching platform.

FIG. 32 shows photographs indicating expression repression by binding of OFF-side factor to the expression ON/OFF switching platform (EYFP fluorescence observation).

FIG. 33 shows photographs of EYFP fluorescence observation of gene expression ON/OFF switching platform introduced Arabidopsis thalian. In each strain, the upper row is that observed from the leaf side, and the lower row is that observed from the root side.

FIG. 34 shows the construction of Agrobacterium binary vector pRIBAC LacSG-tetPF (fusion vector).

FIG. 35 shows a principle of the expression regulation in the LacSG-tetPF inserted plant strain.

FIG. 36 shows miniaturization of Agrobacterium binary vector pRIBAC LacSG-tetPF.

FIG. 37 shows development of genome recombination station (gene recombination by various CRE/Lox cassettes).

FIG. 38 shows various site-specific recombinant sequences (Development of Genome Recombination Station). The sequences provided in the figure correspond to SEQ ID NOs:7-21, ordered from top to bottom in the figure.

FIG. 39 shows analysis by qPCR of the number of copies of introduced DNA in the expression ON/OFF switching platform introduced BY-2 strain.

FIG. 40 shows frequency distribution of the number of copies of introduced DNA in the ON/OFF switching platform introduced BY-2 strain and Arabidopsis thaliana strain.

FIG. 41 shows an example of expression regulation of the expression ON/OFF switching platform as addition of the experiment (FIG. 32) of verification of the effect of the ON/OFF regulator described in Example 1-(9).

FIG. 42 shows that the ON/OFF switching platform is transmitted to the next generation and the next next generation in the ON/OFF switching platform introduced Arabidopsis thaliana strain (Stabilization of On/Off Switching Platform Across Generations).

FIG. 43 shows EYFP fluorescence observation in gene expression ON/OFF switching platform introduced benthamiana tobacco.

FIG. 44 shows the results of introduction of miniaturized linked pRIBAC LacSG-tetPF into BY-2 cells (gene OFF regulation colony) (Acquisition of Strain Having Both On and Off of Gene Expression by Linking Construction).

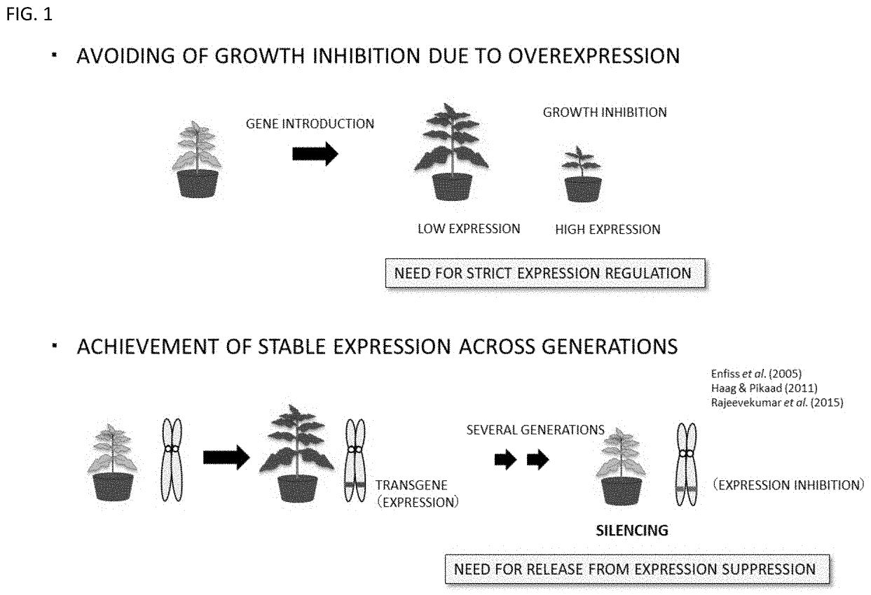

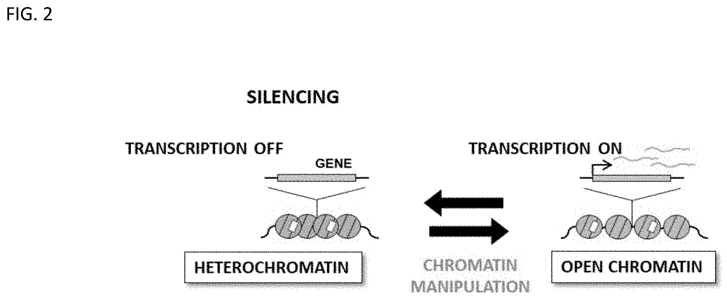

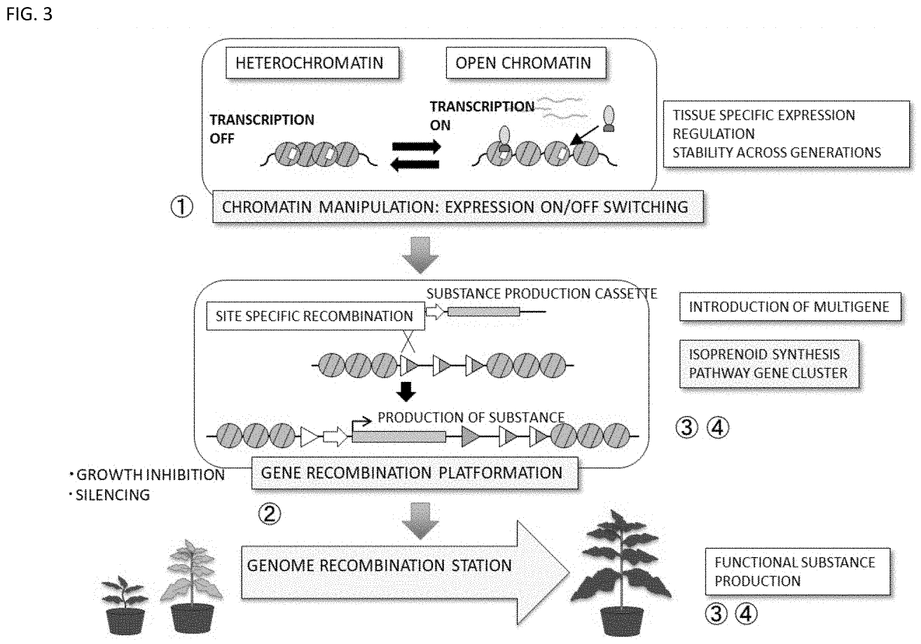

### DESCRIPTION OF THE PREFERRED EMBODIMENTS

### Plant Recombinant Gene Expression Regulatory Platform DNA Sequence

The plant gene expression regulatory platform DNA sequence of the present invention is characterized in that the DNA sequence includes an artificial alphoid DNA sequence (1) and a multigene (a plurality of genes) expression cassette sequence (2) that is a foreign gene and that the artificial alphoid DNA sequence is linked to the upstream (5′ side) and downstream (3′ side) of the expression cassette sequence. Incidentally, the platform DNA sequence may appropriately include, for example, various linker sequences required for linking each component (e.g., gene) during synthesis.

### (1) Artificial Alphoid DNA Sequence

The (1) “artificial alphoid DNA sequence” in the present invention is artificially synthesized based on nucleotide sequence information of an “alphoid DNA sequence” observed in a human chromosome.

Since disruption of mechanism of chromosome segregation causes cell death, overgrowth, disease, and so on, the process thereof needs to be regulated very precisely. The region that plays an essential role in this chromosome segregation is centromere. Nucleosome containing CENP-A that is included in chromatin of the centromere is essential for assembly of proteins specific to the centromere, and in the M phase of cell division, a kinetochore (centromere) structure is formed in centromere chromatin. The kinetochore checks the interaction between centromere and spindle microtubule and regulates chromosome movement. It is now believed that over 100 proteins assemble in this kinetochore structure. In human chromosomes, the centromere is formed in a huge region consisting of a hierarchical repetitive structure ranging from 0.5 Mb to several Mb based on a higher ordered repeat unit composed of a plurality of repeat units of about 170 to 171 bp called alphoid DNA. A DNA sequence called “CENP-B box sequence” is a site on a DNA to which CENP-B protein binds, the CENP-B protein binds to centromere and is involved in kinetochore formation. In many cases, the repeat unit containing the CENP-B box sequence and a repeat sequence not containing the CENP-B box sequence are alternately repeated.

As an example of the alphoid DNA sequence of such a human chromosome, α21-I sequence of human chromosome 21 is mentioned. The centromere region of human chromosome 21 includes the following two regions called α21-I and α21-II.

α21-I: a region of about 1.3 Mbp in which a fundamental unit (171 bp) is repeated 11 times to form a higher ordered repeat unit and this higher ordered repeat unit is further repeated about 700 times to form a well-regulated hierarchical repetitive structure. Incidentally, the nucleotide sequences of the fundamental units are slightly different from each other (homology: about 80% to 90%).

α21-II: a region of 1.9 Mbp or more in which the homology of the fundamental repeat units highly varies and no hierarchical structure is formed, unlike α21-I.

The artificial alphoid DNA sequence of the present invention is characterized in that the DNA sequence has a repeat DNA structure (hierarchical repetitive structure) in the alphoid DNA forming the centromere of a human chromosome and includes a nucleotide sequence a part of which is replaced by a binding site of a gene expression regulator (inducer). The artificial alphoid DNA sequence can include an arbitrary number of the binding sites at arbitrary positions. For example, as shown in Examples, at least a part or the whole of the repeat units constituting the alphoid DNA and not containing the CENP-B box sequence can include the binding site of the gene expression regulator (inducer). Alternatively, a repeat unit including the CENP-B box sequence may include the binding site of the gene expression regulator (inducer).

Incidentally, the respective artificial alphoid DNA sequences linked to upstream (5′ side) and downstream (3′ side) of the expression cassette sequence do not have to include the same nucleotide sequence.

In addition, preferable examples of the artificial alphoid DNA sequence of the present invention include DNA sequences having a several to 16-time, preferably 4 to 16-time, repeated structure of a higher ordered repeat unit consisting of 11-mer of a repeat unit of 171 bp, and having a length of about 30 kb or less, preferably 7.5 to 30 kb.

More specifically, the higher ordered repeat unit consisting of 11-mer of the repeat unit constituting the artificial alphoid DNA sequence is the following DNA sequence:

a DNA sequence (1) consisting of the nucleotide sequence of SEQ ID NO: 1 (including tetO) or SEQ ID NO: 2 (including LacO);

a DNA sequence (2) that hybridizes with a DNA sequence consisting of a nucleotide sequence complementary to the nucleotide sequence of (1) under stringent conditions, the DNA sequence (2) consisting of a nucleotide sequence that includes a binding site of a gene expression regulator (inducer) and having substantially the same plant recombinant gene expression regulatory function as that of the DNA sequence (1); or

a DNA sequence (3) that consists of a nucleotide sequence showing a sequence identity of 80% or more, preferably 90% or more, further preferably 95% or more, and most preferably 99% or more to the whole DNA nucleotide sequence of (1) and including a binding site of a gene expression regulator (inducer), the DNA sequence (3) having substantially the same plant recombinant gene expression regulatory function as that of the DNA sequence (1).

SEQ ID NO: 1: tetO 11-mer (1886 bp) is a variant in which the sequence of a 11-mer unit (higher ordered repeat) constituted of type I alphoid (sequence having CENP-B box and functioning as centromere) of human chromosome 21 centromere is modified so as to include a tetracycline operator sequence (tetO). The positions of “NheI site: restriction enzyme recognition sequence (GCTAGC)”, “SpeI site: restriction enzyme recognition sequence (ACTAGT)”, and the nucleotide sequences of “tetO” and “CENP-B box” included in SEQ ID NO: 1 are as follows. 1-6 NheI site; 153-171 tetO; 324-342 tetO; 493-509 CENP-B box; 665-683 tetO; 835-851 CENP-B box; 1006-1024 tetO; 1178-1194 CENP-B box; 1350-1368 tetO; 1520-1536 CENP-B box; 1692-1710 tetO; 1862-1878 CENP-B box; and 1881-1886 SpeI site.

SEQ ID NO: 2: LacO 11-mer (1886 bp) is a variant in which the sequence of a 11-mer unit (higher ordered repeat) constituted of type I alphoid (sequence having CENP-B box and functioning as centromere) of human chromosome 21 centromere is modified so as to include a lactose operator sequence (LacO). The positions of “NheI site: restriction enzyme recognition sequence (GCTAGC)”, “SpeI site: restriction enzyme recognition sequence (ACTAGT)”, and the nucleotide sequences of “lacO” and “CENP-B box” included in SEQ ID NO: 2 are as follows.

1-6 NheI site; 147-170 lacO; 318-341 lacO; 493-509 CENP-B box; 659-682 lacO; 835-851 CENP-B box; 1000-1023 lacO;
1178-1194 CENP-B box; 1344-1367 lacO; 1520-1536 CENP-B box; 1686-1709 lacO; 1862-1878 CENP-B box; and 1881-1886 SpeI site.

Hybridization can be performed according to a method known in the art or a method equivalent thereto, such as the method described in Current protocols in molecular biology (edited by Frederick M. Ausubel, et al., 1987) or the method described in Molecular cloning, third ed. (Cold Spring Harbor Lab. Press, 2001). In addition, when a commercially available library is used, hybridization can be performed according to the method described in the attached instruction manual.

In the present specification, the conditions of “hybridization under stringent conditions” when hybridizes are specifically, for example, conditions in which incubation is performed at 42° C. in 50% formamide, 5×SSC (150 mM sodium chloride, 15 mM trisodium citrate, 10 mM sodium phosphate, 1 mM ethylenediaminetetraacetic acid, pH 7.2), 5×Denhardt's solution, 0.1% SDS, 10% dextran sulfate, and 100 μg/mL denatured salmon sperm DNA and then the filter is washed at 42° C. in 0.2×SSC.

Accordingly, examples of the DNA that can hybridize with a DNA including the nucleotide sequence represented by SEQ ID NO: 1 or SEQ ID NO: 2 include DNAs containing a nucleotide sequence of which the degree of identity with the entire nucleotide sequence of the DNA is about 80% or more, preferably 90% or more, further preferably 95% or more, and most preferably 99% or more on average.

In order to determine the sequence identity between two nucleotide sequences, the sequences are pretreated to the optimum state for comparison. For example, a gap is inserted into one sequence to optimize the alignment of the other sequence. Subsequently, the nucleotides at each site are compared to each other. When the nucleotide at a site in a first sequence and the nucleotide at the corresponding site in a second sequence are the same, these sequences are identical to each other at this site. The sequence identity between two sequences is shown by percent of the number of sites at which the sequences are identical with respect to the total number of sites.

According to the above principle, the sequence identity between two nucleotide sequences is determined by, for example, the algorithm of Karlin and Altschul (Proc. Natl. Acad. Sci. USA, 87: 2264-2268, 1990 and Proc. Natl. Acad. Sci. USA, 90: 5873-5877, 1993). The BLAST program or the FASTA program using such an algorithm is mainly used to search a database for a sequence having a high sequence identity to the given sequence. These programs are available, for example, on the website of the US National Center for Biotechnology Information on the internet.

In synthesis of the above-described artificial alphoid DNA sequence, a binding site of a gene expression regulator is inserted into at least a part of the repeat unit constituting the hierarchical repetitive structure and not containing the CENP-B Box sequence. The chromatin structure is transformed between the heterochromatin and the open chromatin by repressing or promoting the binding of the gene expression regulator to the site.

Here, as the “binding site of gene expression regulator (inducer)”, arbitrary DNA sequences including transcriptional regulatory elements known to those skilled in the art, for example, operators such as tetO and LacO can be mentioned. To the transcriptional regulatory element, for example, a sequence-specific DNA binding protein specifically including a repressor such as tetR and rtetR and a transcriptional factor (protein) such as Lac inducer (LacI) binds. Such binding can be repressed or promoted by an appropriate compound in a chemical induction system, such as an estradiol induction system, a tetracycline induction system, IPTG (isopropyl-β-galactopyranoside) induction system, and dexamethasone system.

Meanwhile, as the “gene expression regulator (inducer)” of the present invention, proteins having an activity of inducing transcription of the chromatin structure, for example, human or plant-derived various histone-modifying enzymes (methylation enzyme, deacetylation enzyme, and acetylation enzyme such as those mentioned in Table 1) known to those skilled in the art can be mentioned. Such a gene expression regulator is expressed as a fusion protein with a sequence-specific DNA binding protein such as the transcriptional factor. The fusion protein specifically binds to the corresponding binding site through the transcriptional factor or the like, resulting in binding (tethering) of the gene expression regulator to the binding site.

TABLE 1
[Candidate of gene to be used for expression ON/OFF regulation]

| | Modification and | | | | |
|---|---|---|---|---|---|
| | effect to be | | | | Protein to be fused |
| | introduced | Human gene | Plant gene | ON/OFF | (ER is also added) |
| Close (OFF) side factor | H3K9me3 | SUV39H1 MTD | SUVR4 | OFF | tetR |
| (heterochromatin) | | tTS | | | |
| | H3K9me2 | G9a | SUVH4/KYP | OFF | tetR |
| | H3K27me3 | EZH2 | CLN | OFF | tetR |
| | H3K27me2 | | SWN | | |
| | Deacetylation | HDAC1 | HDA9 | OFF | tetR |
| | | | HDA19 | | |
| Open (ON) side factor | H3K36me3 | ASH1L (MTD) | ASHH2/ESF | ON | rtetR, LacI |
| (euchromatin) | H3K4me3 | MLL | ATXR3/SDG2 | ON | rtetR, LacI |
| | H3K14ac | KAT7 | HAM2 | ON | rtetR, LacI |
| | H4K5ac | | (KAT7 homolog) | | |
| | H4K8ac | | | | |
| | H4K12ac | | | | |
| | H3K9ac H3K14ac | PCAF | GCN5/HAG1 | ON | rtetR, LacI |
| | H3K27ac | P300 | | | rtetR, LacI |
| | Transcription | tTA | There is an | ON | rtetR, LacI |
| | activation | | example of use | | |
| | | | in plant. | | |

That is, respectively, a genetically modified plant body obtained on a medium containing an appropriate amount of a compound, such as estradiol, tetracycline, IPTG, or dexamethasone, is grown under the appropriate conditions to apply the compound to the plant body; thereby the binding of a gene expression regulator to a binding site included in an artificial alphoid DNA sequence is repressed or promoted to regulate the action (e.g., enzyme activity such as acetylation, deacetylation, and methylation of histone and a variety of transcription activation) of the gene expression regulator; and as a result, a change in the chromatin structure (transcription of the chromatin structure between heterochromatin and open chromatin) is induced to regulate the expression of a transgene (recombinant gene) in the recombinant plant body, such as repression of gene expression by heterochromatinization or enhancement of gene expression by open chromatinization.

### (2) Multigene (a Plurality of Genes) Expression Cassette Sequence

The (2) “multigene (a plurality of genes) expression cassette sequence” of the present invention is a DNA sequence that is introduced into the genetically modified plant body and is for inserting a multigene (a plurality of genes) as a target (recombinant gene) to be expressed in the resulting genetically modified plant body. Although the number and the types of genes that can be inserted into the cassette sequence are not particularly limited, usually, several to ten-and-several genes or a multigene having a length of 5 to 100 kb can be introduced into the cassette sequence.

Such a multigene can be inserted into the cassette sequence by an arbitrary method known to those skilled in the art. For that, the cassette sequence preferably includes in advance an arbitrary recombinant enzyme site known to those skilled in the art. Preferable examples of the recombinant enzyme site include VCre/VloxP and SCre/SloxP, which are one type of a Cre/loxP system site-specific recombinant system, as described in Japanese Patent Nos. 5336592 and 5336676.

When all genes of a biosynthesis pathway of a certain compound in a plant body are collectively introduced as an example of the multigene, it is possible to cooperatively increase the expression of these genes, and as a result, the efficiency of biosynthesis of the compound can be increased. Alternatively, in order to verify, for example, introduction of the cassette sequence into a chromosome, the cassette sequence can include an arbitrary marker gene or reporter gene known to those skilled in the art, such as a fluorescent protein gene and a drug selection gene. Incidentally, when a multigene is inserted into the cassette sequence, a promoter (preferably constitutive promoter), a terminator, and other various transcriptional regulatory elements (regulatory sequences) known to those skilled in the art are also preferably inserted into the upstream or downstream of each gene.

Incidentally, in order to obtain a DNA sequence including a multigene (a plurality of genes), an arbitrary method known to those skilled in the art, for example, solid-phase DNA sequential ligation (PRESSO) method (Takita, E., et al., DNA Research, 2013, 20(6), 583-592 doi:10.1093/dnares/dst032, Precise sequential DNA ligation on a solid substrate: solid-based rapid sequential ligation of multiple DNA molecules) can be mentioned.

### DNA Sequence for Expressing Gene Expression Regulator

Furthermore, the present invention also relates to, as described in the following Examples, a DNA sequence for expressing a gene expression regulator including a gene (switching inducible gene) encoding the above-described gene expression regulator. The DNA sequence for the expression preferably encodes multiple types of gene expression regulators and thereby can more efficiently transform the chromatin structure between heterochromatin and open chromatin in the platform DNA sequence.

Incidentally, when each of the genes encoding expression regulators is inserted into the above-mentioned DNA sequence for expression, a promoter (preferably constitutive promoter) and other various transcriptional regulatory elements known to those skilled in the art are also preferably inserted into the upstream of each gene. Furthermore, for example, various linker sequences required for linking each component (e.g., gene) may be included. Plant recombinant gene expression regulatory platform vector including plant recombinant gene expression regulatory platform DNA sequence, gene expression regulator vector including DNA sequence for expressing gene expression regulator, and vector set consisting of these vectors

The present invention also relates to a plant recombinant gene expression regulatory platform vector including a plant recombinant gene expression regulatory platform DNA sequence, a gene expression regulator vector including a DNA sequence for expressing a gene expression regulator, and a vector set consisting of these vectors.

As described in detail also in the following Examples, for example, a gene expression regulator expressed as a fusion protein of a histone-modifying enzyme and a transcriptional factor has a risk of losing the expression thereof due to various reasons. Accordingly, as a preferable example of the gene expression regulator vector, a system (self-activating type) that can self-activate the expression of a gene expression regulator from a DNA sequence for expressing the gene expression regulator such that the gene expression of the gene expression regulator introduced in a gene expression regulator vector is not turned OFF can be mentioned.

In the gene expression regulator vector of the self-activating system, an artificial alphoid DNA sequences included in the plant recombinant gene expression regulatory platform DNA sequence of the present invention is linked to the upstream (5′ side) and downstream (3′ side) of the DNA sequence for expressing the gene expression regulator.

That is, as a binding site of the gene expression regulator included in the artificial alphoid DNA sequence, for example, an LacO sequence, which is a transcriptional regulatory element, is inserted; and a gene encoding LacI-fusion (ON), which is a fusion protein of a gene expression regulator (ON) (e.g., histone-acetylating enzyme) and a Lac inducer (LacI), which is a transcriptional factor (protein), is inserted into a DNA sequence for expressing the gene expression regulator. Furthermore, the expression of the LacI-fusion (ON) from a cassette sequence itself surrounded by the LacO alphoid DNA can constantly express gene expression regulators such as tetR-fusion (OFF) and rtetR-fusion (ON) included in the same cassette sequence, in addition to the LacI-fusion (ON).

Furthermore, it is also possible that one vector includes all of the following elements included in the above-mentioned vectors, that is, a plant recombinant gene expression regulatory platform DNA sequence and a DNA sequence for expressing the gene expression regulator and, further, preferably an artificial alphoid DNA sequence necessary for a self-activating type gene expression regulator vector. As a result, a “plant gene recombinant fusion vector” having such a configuration acts as a vector having both functions of a plant recombinant gene expression regulatory platform vector and a gene expression regulator vector.

Genes (vectors) can be introduced by a single manipulation by using such an inducible vector. Furthermore, there are advantages that even if a recombinant gene on a plant recombinant gene expression regulatory platform DNA sequence is harmful to growth of plants, gene expression on the platform DNA sequence can be repressed from the beginning of Agrobacterium infection by using the drug selection gene present in the DNA sequence for expressing the gene expression regulator.

There are not restrictions on the type, origin, etc. of the vector of the present invention, and the vector can be appropriately selected depending on, for example, the methods for growing the vector and introducing the vector into a plant body mentioned below. For example, a plasmid vector is preferable. Furthermore, as appropriate, any other vector component known to those skilled in the art, such as replication origin, various restriction enzyme sites, and RB and LB sequences, can be included.

Based on the above description and common general technical knowledge in the art, the plant gene expression regulatory platform DNA sequence, DNA sequence for expressing the gene expression regulator, and vectors including them of the present invention can be easily designed and prepared by those skilled in the art.

### Genetically Modified Plant Body and Method for Manufacturing it

As the methods for introduction and transformation of the vectors to a plant body (including plant cells and a part of the plant body, such as tissues represented by seeds and leaves), methods known to those skilled in the art, such as an Agrobacterium method, a particle gun method, and a whisker method, can be mentioned depending on, for example, the type of the plant body. The type of the plant body is not particularly limited, and examples thereof include Cruciferous plants such as Arabidopsis thaliana, Solanaceae plants such as tobacco, and other gramineous plants with high practical values, such as rice.

The Agrobacterium method is a method using a Ti plasmid included in Agrobacterium such as Agrobacterium tumefaciens (formal name: Rhizobium radiobacter) and Agrobacterium rhizogenes (formal name: Rhizobium rhizogenes) and is superior to other methods in terms of particularly its economic efficiency and convenience. A target gene (T-DNA) is often introduced into the nuclear genome of a plant with a low number of copies of about 1 or 2. In the Agrobacterium method, an intermediate vector method and a binary vector method are included. Currently, the binary vector method is the mainstream, and the vector to be used in the method is called Agrobacterium binary vector.

In the binary vector method, a vir helper Ti plasmid lacking the original T-DNA but retaining the vir region which is a gene cluster required for introducing the T-DNA into a plant genome and a strain of A. tumefaciens holding the plasmid have been developed. Since a gene cluster necessary for gene introduction (chv genes: chromosomal virulence genes) into a plant is present also on the chromosome of of A. tumefaciens, A. tumefaciens is further required in the Agrobacterium method also as a host for the Ti plasmid. There are short sequences called RB (right border: right border sequence) and LB (left border: left border sequence) at both ends of the T-DNA. The sequence sandwiched between the RB and the LB is introduced into a plant, and this sequence does not have specificity. Accordingly, an arbitrary artificial T-DNA can be constructed by inserting a gene to be introduced into a plant and a selection marker gene for selecting a transgenic plant into between the RB and the LB. The action relationship between the vir region and the T-DNA is trans, and both need not be present on the same plasmid. Accordingly, a T-DNA plasmid (binary vector) that is an easily-operable small shuttle vector containing an artificial T-DNA is amplified in, for example, Escherichia coli and is then introduced into an A. tumefaciens strain retaining the vir helper Ti plasmid, and the thus obtained A. tumefaciens is infected to a plant.

In the method of the present invention, the plant body transformed by the vector can be appropriately selected by a method according to the type of the selection marker gene included in the vector. For example, when a hygromycin resistance gene is included, a transgenic plant body can be easily selected by cultivating plants using a medium containing hygromycin under suitable conditions.

The transformation method above can manufacture a genetically modified plant body containing a plant recombinant gene expression regulatory platform DNA sequence of the present invention and a DNA sequence for expressing the gene expression regulator of the present invention introduced into a chromosome. Accordingly, the present invention also relates to such a manufacturing method. Incidentally, the platform DNA sequence and the cassette sequence may be introduced at any position on the plant chromosome and may be introduced, for example, into different chromosomes, respectively.

### Method for Regulating Expression of Recombinant Gene in Genetically Modified Plant Body

Repression or promotion of binding of a gene expression regulator to a binding site included in the artificial alphoid DNA sequence in the plant recombinant gene expression regulatory platform DNA sequence of the present invention induces transformation of the chromatin structure between heterochromatin and open chromatin on the artificial alphoid DNA sequence and further on the multigene (a plurality of genes) expression cassette sequence through the action of the gene expression regulator, for example, acetylation and methylation of histone. As a result, expression of the recombinant gene, multigene (a plurality of genes), between the artificial alphoid DNA sequences can be regulated. As already described, the binding of a gene expression regulator to a binding site included in an artificial alphoid DNA sequence can be repressed or promoted by allowing an appropriate compound depending on a chemical induction system to be used to act on a plant body (for example, allowing a plant body to grow in a medium containing the compound) by an arbitrary method or means known to those skilled in the art.

Incidentally, the plant recombinant gene expression regulatory platform DNA sequence, the DNA sequence for expressing the gene expression regulator, and the vectors including these sequences described above can be appropriately synthesized using a gene engineering procedure or means known to those skilled in the art as described in Examples based on the present specification and common general technical knowledge in the art.

The present invention will now be described in detail according to Examples. Incidentally, the Examples are merely aspects, and the technical scope of the present invention is not limited by the description of these Examples and is determined by the description of the entire specification, the description contents of the references and the like cited in the present specification, and the common general technical knowledge in the art.

Incidentally, unless otherwise specified in the present specification, various conditions, means, procedures, etc. in the Examples and so on can be appropriately set and implemented by those skilled in the art in accordance with common general technical knowledge in relevant technical field.

### EXAMPLES

### 1. Production of Plant Recombinant Gene Expression Regulatory Platform DNA Sequence (Expression ON/OFF Switching Platform)

### 1-(1) Structure of Gene Expression ON/OFF Switching Platform

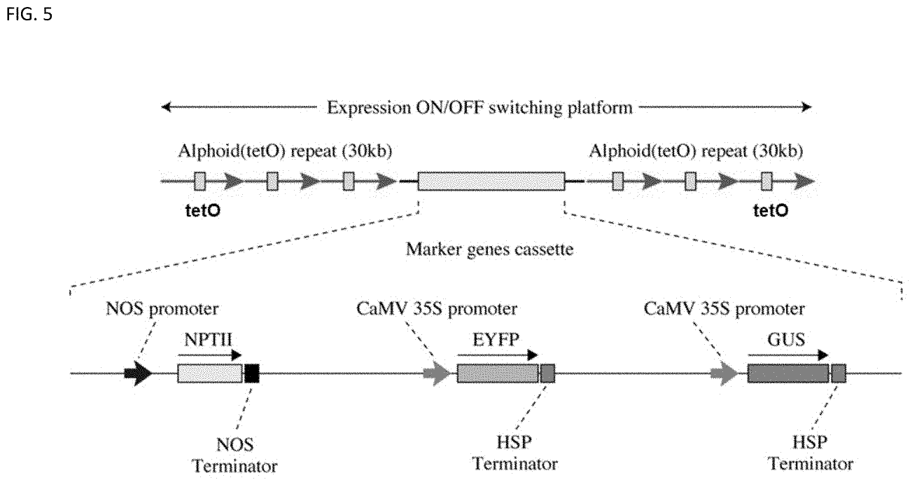

A gene expression ON/OFF switching platform (TAC vector #1, TAC vector, see: Liu, Y. G., Shirano, Y., Fukaki, H., Yanai, Y., Tasaka, M., Tabata, S. and Shibata, D., Complementation of plant mutants with large genomic DNA fragments by a transformation-competent artificial chromosome vector accelerates positional cloning, Proc. Natl. Acad. Sci. USA, 1999, 96, 6535-6540; and Shibata, D. and Liu, Y. G., Agrobacterium-mediated plant transformation with large DNA fragments, Trands in Plant Science, 2000, 5, 354-357) has a structure in which a synthetic alphoid DNA (tetO alphoid DNA) of 30 kb consisting of 171 bp repeat units repeated sandwiches from both sides of a marker gene cassette consisting of three gene expression markers (NPTII, EYFP, and GUS) arranged in the forward direction (FIG. 5). This switching platform was constructed for the purpose of changing the chromatin structure in the adjacent region by manipulating the chromatin structure on the tetO alphoid DNA with a tetR fusion protein and therethrough regulating ON/OFF of the gene expression on the marker gene cassette at the center sandwiched by the tetO alphoid DNA.

Three genes, a drug-selection marker NPTII gene, a gene encoding an EYFP protein suitable for fluorescence observation with a microscope, and a gene encoding a GUS protein suitable for plant tissue staining, are located on the marker gene cassette sequence. A promoter and a terminator are located upstream and downstream of these genes, respectively, to form a gene expression cassette (FIG. 5).

### 1-(2) Principle of Expression ON/OFF Switching System

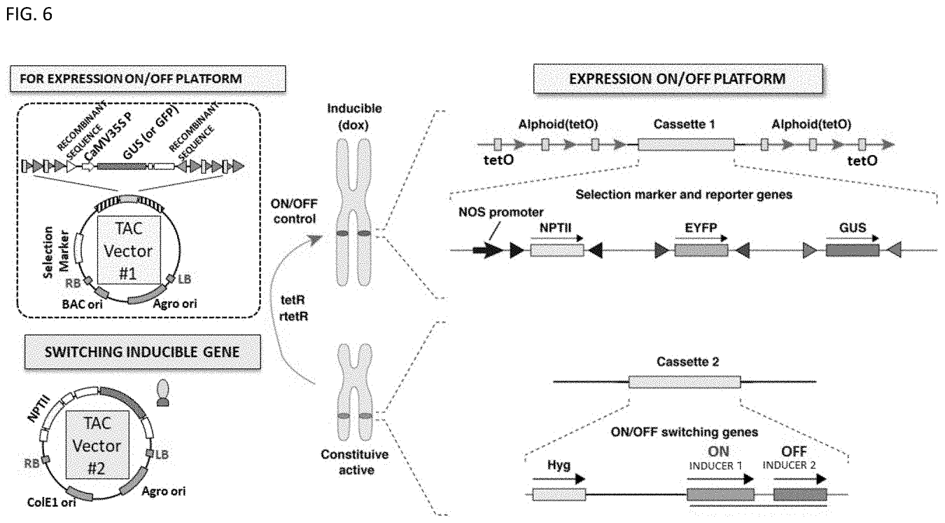

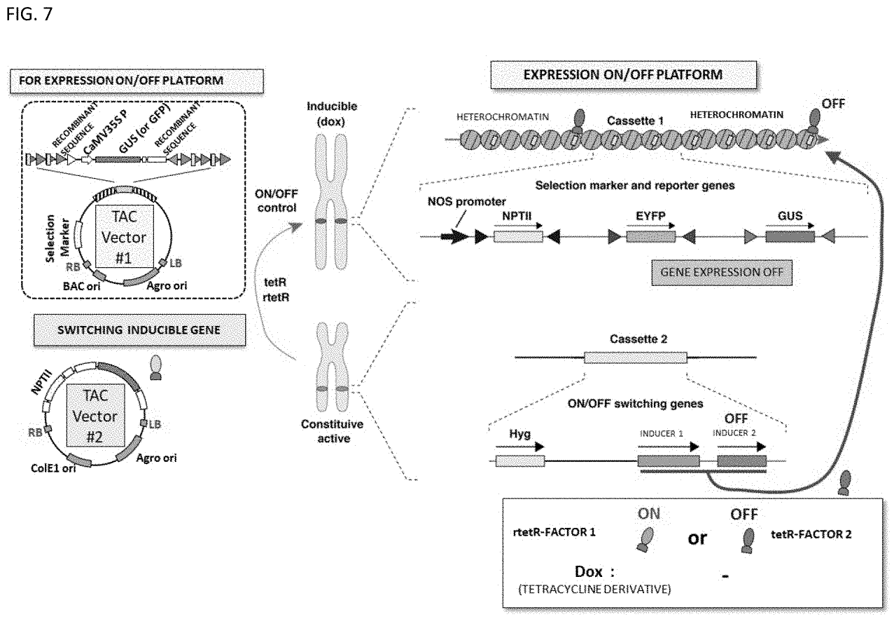

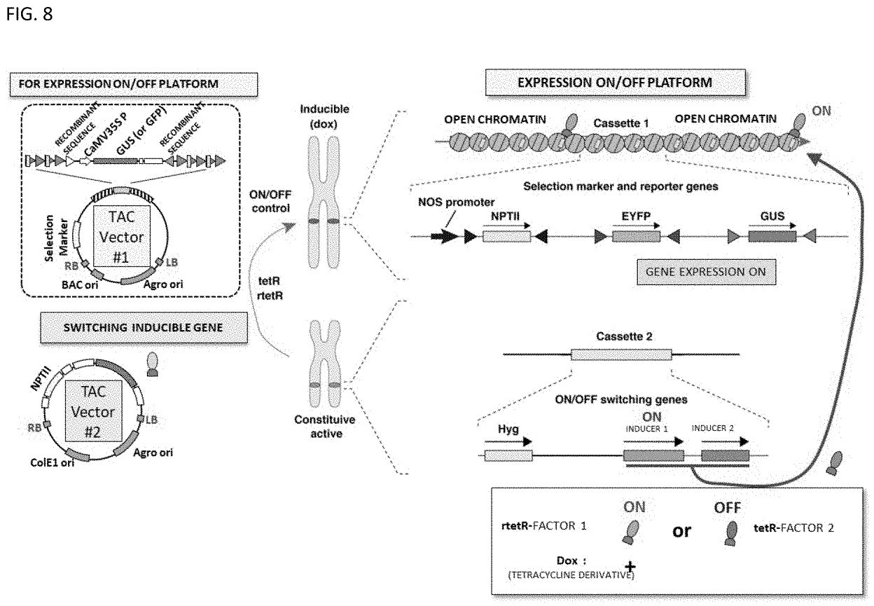

The expression ON/OFF switching platform is realized by insertion of the T-DNA into a plant genome with the Agrobacterium binary vector (TAC vector #1) (FIG. 6). The expression ON/OFF switching platform aims to perform the expression ON/OFF regulation of the gene region embedded in the central region by manipulating the chromatin structure on the tetO alphoid DNA, regardless of the chromatin structure of the genome insertion site. This regulation of the chromatin structure on the tetO alphoid DNA utilizes the tetO sequence embedded in the tetO alphoid DNA at a frequency of one per about 340 bp. It is possible to bind (tethering) an arbitrary protein (gene expression regulator) fused with tetR to this tetO sequence (FIG. 6). In addition, the binding of tetR to tetO is lost by addition of tetracycline or a derivative thereof (FIGS. 7 and 8).

In contrast, a reverse tetracycline repressor (rtetR), which is a mutant of tetR, strongly binds to tetO by addition of tetracycline or a derivative thereof (FIGS. 7 and 8). The opposite properties of tetR and rtetR are also useful for ON/OFF regulation of gene expression. Specifically, regulation is possible in such a manner that the gene expression is OFF when tetracycline and derivatives thereof are not added and is ON when they are added by expressing a fusion protein (tetR-fusion (OFF)) of tetR and a factor (including a histone-modifying enzyme or the like) repressing gene expression and a fusion protein (rtetR-fusion (ON)) of rtetR and a factor (a factor involved in, for example, transcription activation) promoting gene expression (FIGS. 7 and 8). A gene cassette expressing these tetR and rtetR fusion factors is introduced into a cell with an Agrobacterium binary vector (TAC vector #2) that is different from the expression ON/OFF switching platform construction (TAC vector #1), and a regulator is expressed independently from the gene expression on the expression ON/OFF switching platform (FIGS. 6, 7, and 8).

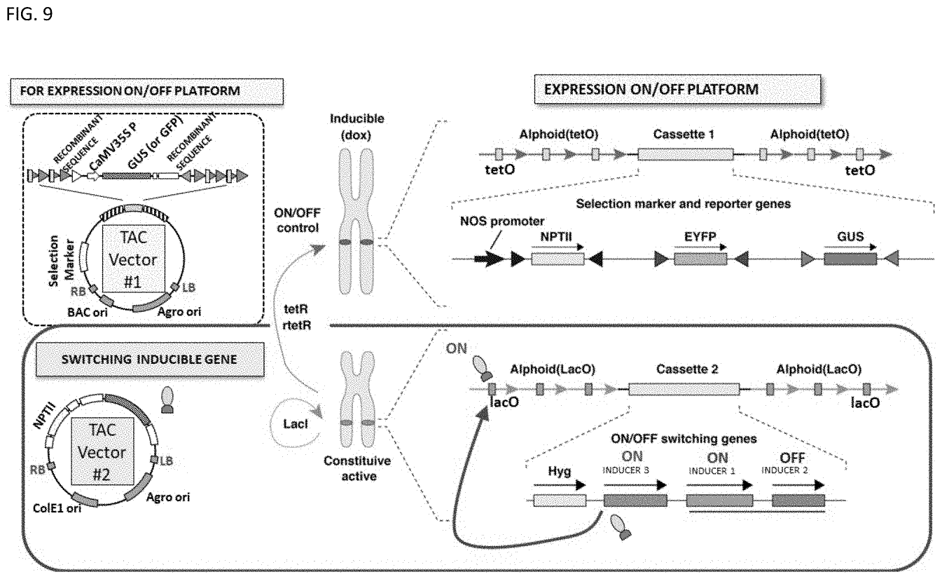

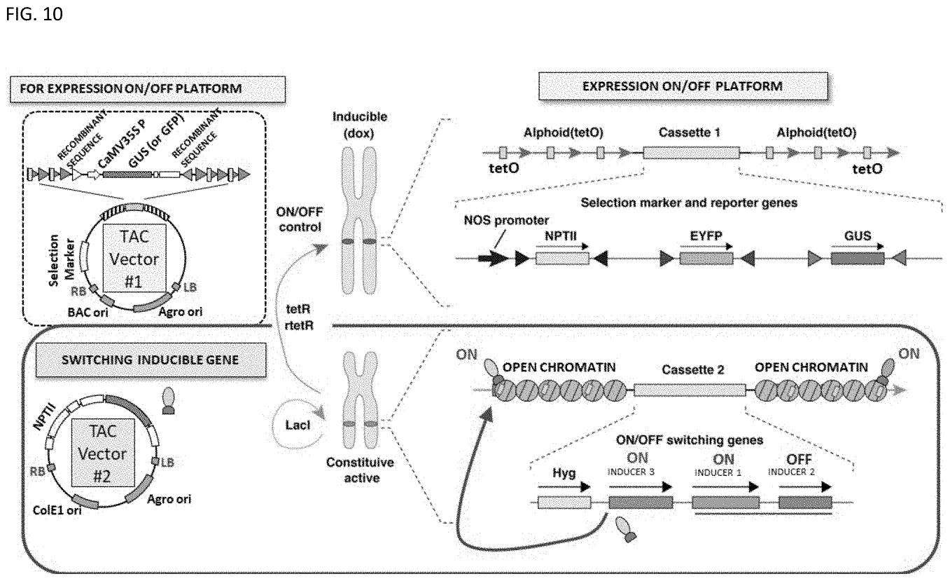

Furthermore, there is a risk of losing the expression of a gene expression regulator by the reasons already described. If the expression of the regulator is turned OFF, the original purpose, gene expression regulation on the platform, cannot be performed. Accordingly, a system that can self-activate the gene expression from a DNA sequence (regulator cassette) for expressing the gene expression regulator so that the gene expression from the regulator cassette is not turned OFF (FIGS. 9 and 10). Here, the LacO sequence is a binding sequence of the Lad protein, and it is possible to bind a factor (LacI-fusion (ON)) that regulates the gene expression of histone acetyltransferase or the like fused with Lad to ON to the LacO alphoid DNA. Furthermore, the LacI-fusion (ON) is allowed to be expressed from the gene cassette itself surrounded by the LacO alphoid DNA, as a result, not only the LacI-fusion (ON) but also the tetR-fusion (OFF) and the rtetR-fusion (ON) included in the same cassette can be constantly expressed.

### 1-(3) TetO Alphoid DNA, LacO Alphoid DNA, and Repeated Extension Thereof

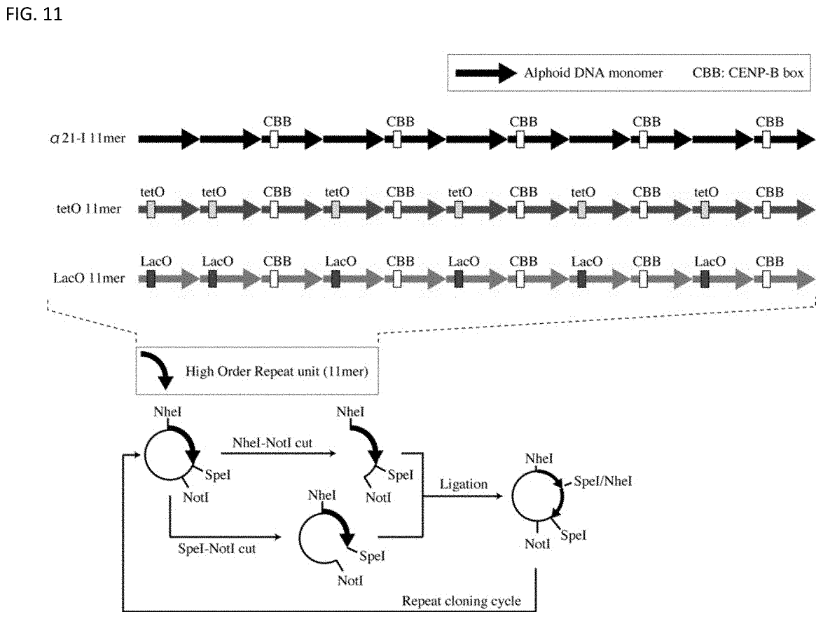

A human centromere sequence, alphoid DNA, is a high-frequency repeat sequence. The human chromosome 21 alphoid DNA is constituted of repeat units called α21-I 11-mer. This 11-mer sequence has a structure in which 171-bp sequences having slightly different sequences called alphoid monomers are connected 11 times. Five of these alphoid monomers include a binding sequence of CENP-B, which is one of human centromere proteins, a CENP-B box sequence (the upper row of FIG. 11).

The tetO 11-mer is a sequence in which a tetracycline operator sequence (tetO), which is a binding sequence of the tetracycline repressor (tetR), is embedded in a monomer not including the CENP-B box in the α21-I 11-mer, and was produced by DNA artificial synthesis (the upper row of FIG. 11, SEQ ID NO: 1).

The LacO 11-mer is a sequence in which a Lac operator sequence (LacO), which is a binding sequence of a Lac repressor (LacI), is embedded instead at the position of the tetO in the tetO alphoid. This sequence was also produced by DNA artificial synthesis (the upper row of FIG. 11, SEQ ID NO: 2).

NheI, SpeI, and NotI sites were added to ends of these alphoid 11-mer sequences and were cloned in a cloning vector pBS KMneo (SEQ ID NO: 3). Since the NheI and SpeI sites among them have the same cohesive end shape when the DNA is cleaved, linking using a DNA ligase is possible. The site formed by hetero-linkage of the sites cleaved with NheI and SpeI is no longer recognized by either NheI or SpeI and is not cleaved. By using this property, a method for repeatedly extending the insert between NheI and SpeI by twice has been established (Ohzeki, J, Nakano, M., Okada, T., and Masumoto, H., CENP-B box is required for de novo centromere chromatin assembly on human alphoid DNA, J. Cell Biol., 2002, Dec. 9, 159(5): 765-75; and Okamoto, Y., Nakano, M., Ohzeki, J., Larionov, V., and Masumoto H., A minimal CENP-A core is required for nucleation and maintenance of a functional human centromere, EMBO J., 2007, Mar. 7, 26(5): 1279-91).

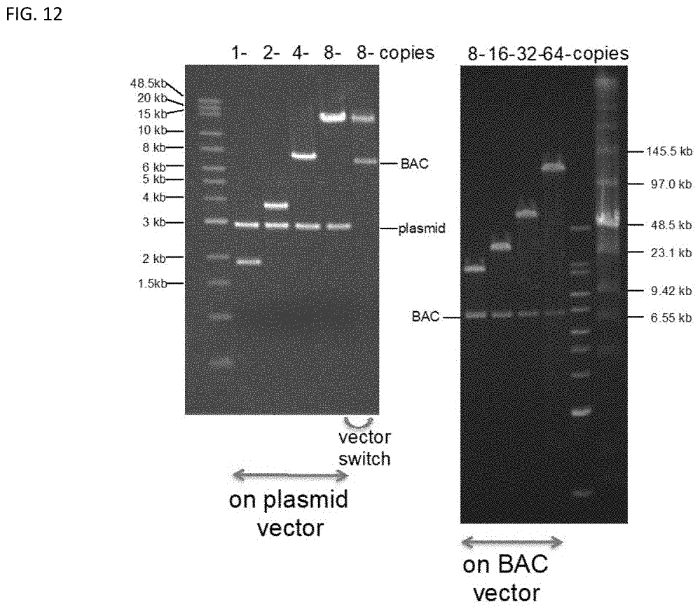

For example, when the NheI-NotI fragment and the SpeI-NotI fragment in the lower row in FIG. 11 are linked to each other, although the number of repeat inserts is doubled, the positional relationship of the restriction enzyme sites does not change. Accordingly, it is possible to repeatedly extend the insert twice by repeating similar restriction enzyme treatment and ligation reaction. By applying the method, a BAC vector in which the respective alphoid 11-mer sequences of the tetO-11-mer and the LacO-11-mer were each repeated 16 times was produced (FIG. 12).
1-(4) Production of Agrobacterium Binary Vector pRIBAC

It is generally known that the repeat sequence cloned on an Escherichia coli plasmid is easily deleted by a recombination reaction. Accordingly, in order to prevent deletion and stably maintain a repeat sequences such as a human alphoid DNA, it is important to keep the number of copies of the plasmid per cell as low as possible.

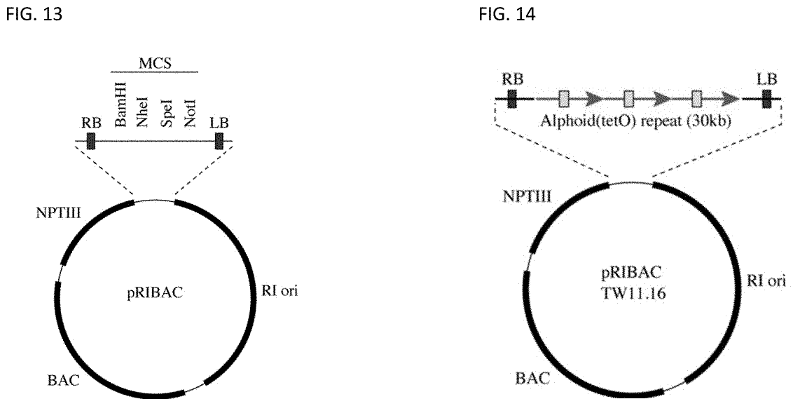

Accordingly, in order to stably retain a plasmid containing the alphoid DNA in Escherichia coli and Agrobacterium, the BAC vector sequence that is maintained at one copy per Escherichia coli cell and the replication origin (Ri ori sequence) in Agrobacterium were combined to produce a binary vector that was replicated and maintained in both cells (FIG. 13).

The BAC vector sequence was obtained by PCR amplification from a pBAC108L vector (GenBank: U51114.1), and the Ri ori sequence was obtained by PCR amplification from a pRI-201-AN-GUS vector (TAKARA). In addition, an NPTIII gene expression cassette sequence as a selection marker to be used in Escherichia coli and Agrobacterium cells was obtained by PCR amplification from the pRI-201-AN-GUS vector. These sequences were combined to produce a binary vector pRIBAC (SEQ ID NO: 4).

### 1-(5) Construction of Gene Expression ON/OFF Switching Platform Vector

In order to construct a gene expression ON/OFF switching platform DNA sequence of the present invention, a pRIBAC TW11.16 plasmid obtained by inserting a tetO 11-mer repeat sequence (tetO alphoid DNA) having a length of 30 kb into pRIBAC was first produced using an Escherichia coli strain DH10B (FIG. 14). Here, the inserted tetO alphoid DNA fragment includes an NheI-NotI site on an end and an SpeI site between the tetO alphoid DNA and the NotI site. Accordingly, in the pRIBAC TW11.16 obtained by inserting the tetO alphoid DNA into the SpeI-NotI site of pRIBAC, an insert can be inserted between the repeat SpeI-NotI sites by using restriction enzyme treatment and ligation as in FIG. 11.

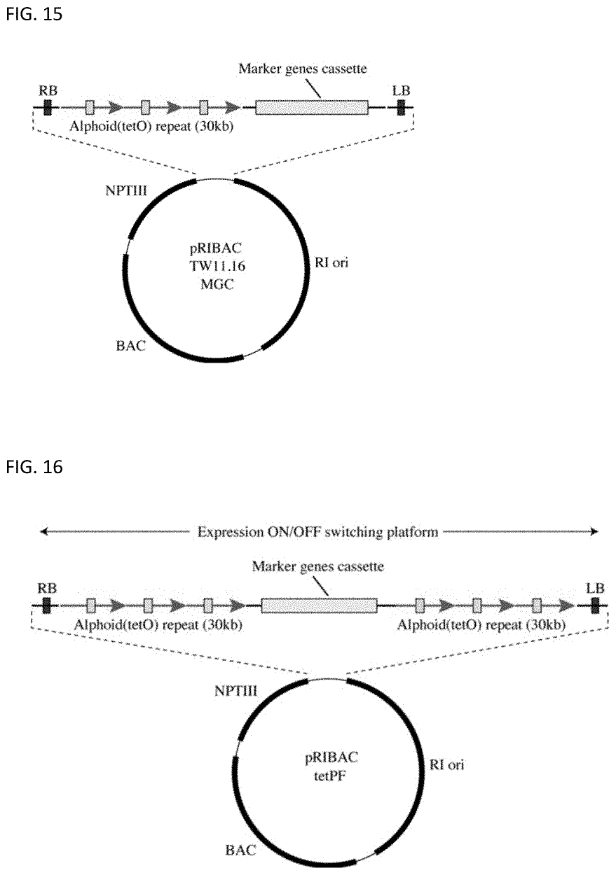

Subsequently, a marker gene cassette sequence (multigene expression cassette sequence) was linked to pRIBAC TW11.16 (FIG. 15). The marker gene cassette sequence is constructed of gene expression cassettes, NPTII, EYFP, and GUS, arranged in the forward direction (FIG. 5). Among the gene expression cassettes, the NPTII and GUS gene cassettes were produced by a PCR method using a pRI-201-AN-GUS vector (TAKARA) as the template, and the EYFP gene was produced by a PCR method using a pJETY3 vector (EMBO J., 2012, May 16, 31 (10), 2391-402) as the template. In addition, the three gene cassettes were linked by ligation using the NheI and SpeI sites having mutually joining ends to form a marker gene cassette sequence consisting of the connected three genes (FIG. 5). This core sequence can be cut out with NheI-SpeI (NotI), and the core sequence cut out with NheI-NotI was inserted into the SpeI-NotI site of pRIBAC TW11.16 to produce a vector, pRIBAC TW11.16 MGC, in which the tetO alphoid DNA and the core sequence (multigene (a plurality of genes) expression cassette sequence) of a genome recombination station were linked to each other (FIG. 15).

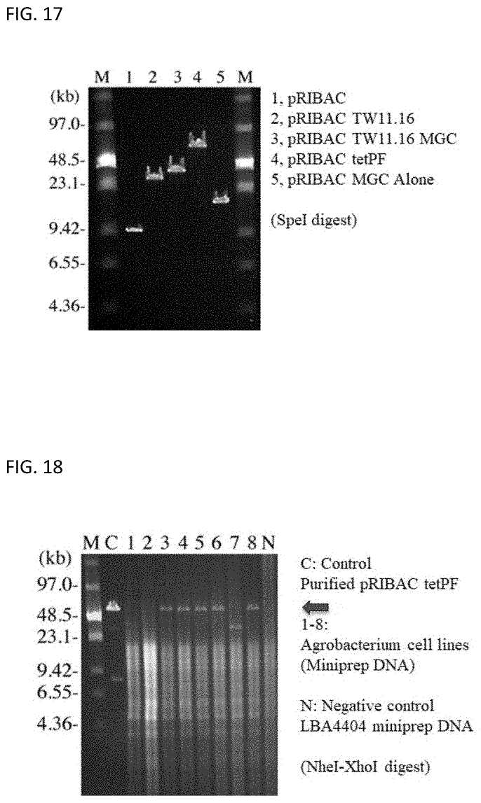

Finally, the tetO alphoid DNA fragment cut out from pRIBAC TW11.16 with NheI and NotI was inserted between the SpeI-NotI sites of pRIBAC TW11.16 MGC to complete a plasmid (pRIBAC tetPF) for producing a gene expression ON/OFF switching platform, a vector including the plant recombinant gene expression regulatory platform DNA sequence of the present invention (FIG. 16). The plasmid DNAs (pRIBAC, pRIBAC TW11.16, pRIBAC TW11.16 MGC, and pRIBAC tetPF) used in the process of producing this pRIBAC tetPF were all produced using an Escherichia coli strain DH10B and were purified with a Large Construction kit (QIAGEN). Subsequently, these plasmids derived from Escherichia coli were cleaved with SpeI, and the sizes thereof were verified by pulsed-field gel electrophoresis (PFGE) (FIG. 17). In addition, a construction (pRIBAC MGC Alone: SEQ ID NO: 5) of only a marker gene cassette not including the alphoid DNA sequence was also produced using an Escherichia coli strain DH10B, and similarly cleavage with SpeI was performed, and the size was verified by PFGE (FIG. 17). Incidentally, the pRIBAC MGC Alone was produced by inserting the marker gene cassette sequence cut out with NheI and NotI between the SpeI-NotI sites of pRIBAC. No abnormalities were observed in the electrophoresis patterns of these plasmids, and no significant deletion of the repeat sequences of the tetO alphoid DNA was observed.

In order to introduce a gene expression ON/OFF switching platform DNA sequence (construction) into a plant cell, it is necessary to introduce the pRIBAC tetPF plasmid to Agrobacterium cells from Escherichia coli to incorporate the pRIBAC tetPF plasmid into a plant genome as a T-DNA. Accordingly, the plasmid was introduced using electrocompetent cells of the Agrobacterium cell line LBA4404 (TAKARA) (introduction conditions: 25 μFm, 200 Ω, 2.4 kV, 0.1 mm cuvette, 20 μL competent cell). Subsequently, Agrobacterium cells into which the pRIBAC tetPF plasmid was introduced were seeded on an LB plate containing kanamycin (30 μg/mL) to form colonies.

In Escherichia coli, the BAC plasmid such as pRIBAC tetPF was maintained at one copy per cell and was unlikely to be recombined. Even if an actually purified plasmid was analyzed by PFGE, no abnormalities were observed in the vector construction (FIG. 18). On the other hand, the stability of the repeat sequences on the plasmid having Ri ori in Agrobacterium cells is not well known. Accordingly, multiple single colonies of Agrobacterium into which the pRIBAC tetPF plasmid was actually introduced were picked up and cultured, and DNA was collected therefrom and was subjected to PFGE analysis.

The plasmid DNA possessed by Agrobacterium was collected by an alkali-SDS method as in Escherichia coli and was treated with restriction enzymes NheI and XhoI, followed by analysis by PFGE (FIG. 18). A band with the same degree of migration as that of the introduced BAC plasmid was observed in a half or more of the analyzed strains to confirm that the repeat sequence incorporated on the plasmid having Ri ori was stably maintained also in Agrobacterium cells.

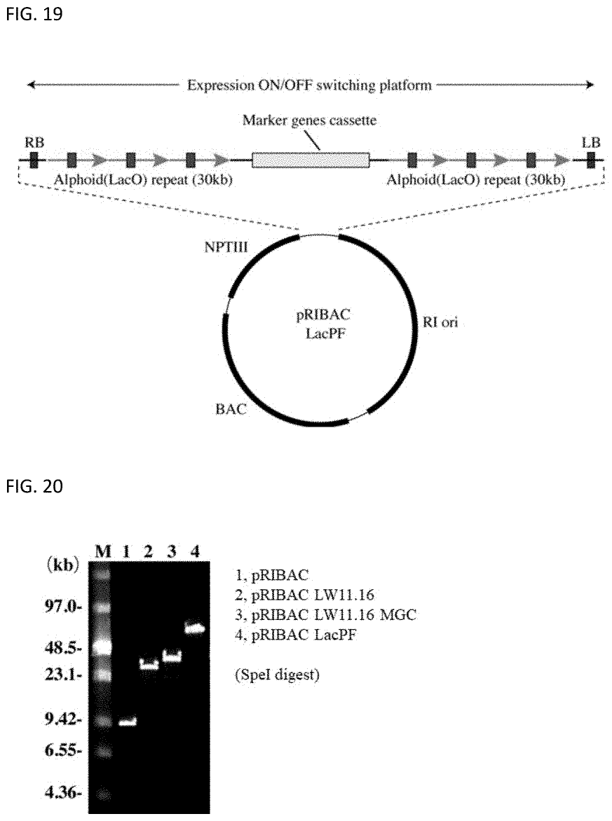

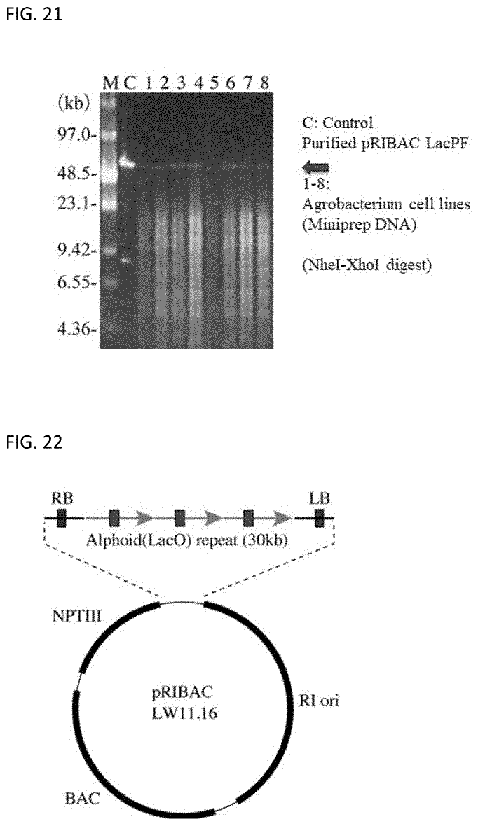

A pRIBAC LacPF plasmid, a vector including the plant recombinant gene expression regulatory platform DNA sequence of the present invention, was also produced by the same method as the pRIBAC tetPF constructed above using the LacO alphoid DNA instead of the tetO alphoid DNA (FIG. 19). The plasmid DNAs (pRIBAC, pRIBAC LW11.16, pRIBAC LW11.16 MGC, and pRIBAC LacPF) used in the process of producing the pRIBAC LacPF were all produced using an Escherichia coli strain DH10B, purified with a Large Construction kit (QIAGEN), and then cleaved with SpeI, and the sizes thereof were verified by pulsed-field gel electrophoresis (PFGE) (FIG. 20). In addition, it was confirmed that the pRIBAC LacPF plasmid introduced into Agrobacterium cells was stably maintained in a half or more of the Agrobacterium strains (FIG. 21).

### 1-(6) Production of Self-Activating Type Regulator Expression Platform Vector

In order to construct the “self-activating type regulator expression platform” vectors (FIGS. 9 and 10) as already described above, a plasmid pRIBAC LW11.16 was first produced using an Escherichia coli strain DH10B by inserting an LacO 11-mer repeat sequence (LacO alphoid DNA) having a length of 30 kb into pRIBAC (FIG. 22). Here, the inserted LacO alphoid DNA fragment includes an NheI-NotI site on an end and an SpeI site between the LacO alphoid DNA and the NotI site. Accordingly, in the pRIBAC TW11.16 obtained by inserting the LacO alphoid DNA into the SpeI-NotI site of pRIBAC, an insert can be inserted between the repeat SpeI-NotI sites (the lower row in FIG. 11).

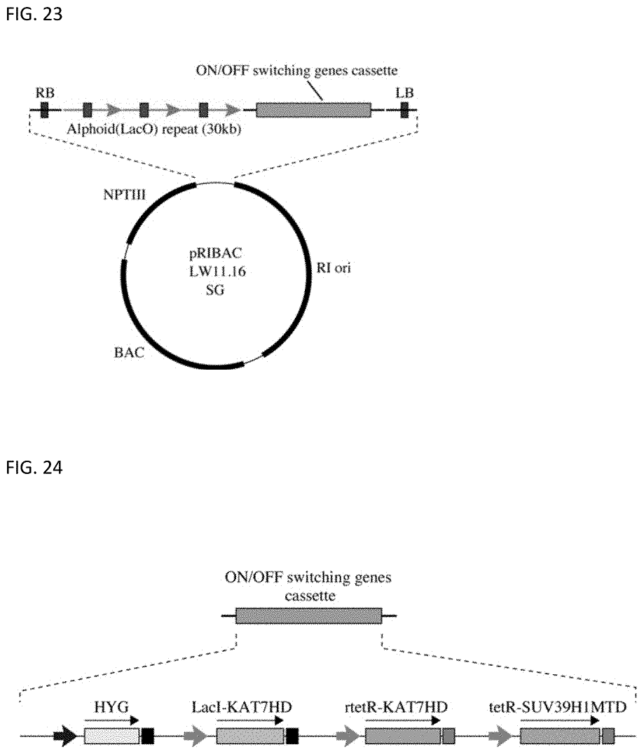

Subsequently, an ON/OFF regulator expression cassette was linked to pRIBAC LW11.16 (FIG. 23, Table 1). In the expression cassette, gene expression cassettes encoding the enzyme domain of a LacI-fused human acetylation enzyme KAT7 (LacI-KAT7HD), the enzyme domain of a rtetR-fused KAT7 (rtetR-KAT7HD), and the enzyme domain of a tetR-fused human methylation enzyme SUV39H1 (tetR-SUV39H1MTD), respectively, are linked in the forward direction, in addition to a drug-selection marker hygromycin (Hyg) resistance gene (FIG. 24, Table 1). This acetylation enzyme KAT7 is a factor regulating the chromatin structure to the direction of activating (promoting) the gene expression (turning on) by acetylating histone, such as H3 and H4, and the methylation enzyme SUV39H1 is a factor regulating the chromatin structure to the direction of repressing the gene expression (turning OFF) by trimethylating the ninth lysine residue of histone H3 (EMBO J., 2012, May 16, 31 (10), 2391-402; and Dev. Cell., 2016, Jun. 6, 37 (5), 413-27). These gene sequences are publicly known and were obtained from the Halo tag fusion library of Kazusa DNA Res. Inst., and only domains having the required enzymatic activity were used for cloning using a PCR method. Each gene cassette was linked by ligation using the NheI and SpeI sites having mutually joining ends to form an ON/OFF regulator expression cassette of the connected four genes (FIG. 23). This cassette sequence can be cut out with NheI-SpeI (NotI), and the cassette sequence cut out with NheI-NotI was inserted into the SpeI-NotI site of pRIBAC TW11.16 to produce a vector, pRIBAC LW11.16 SG, in which the LacO alphoid DNA and the ON/OFF regulator expression cassette were linked to each other.

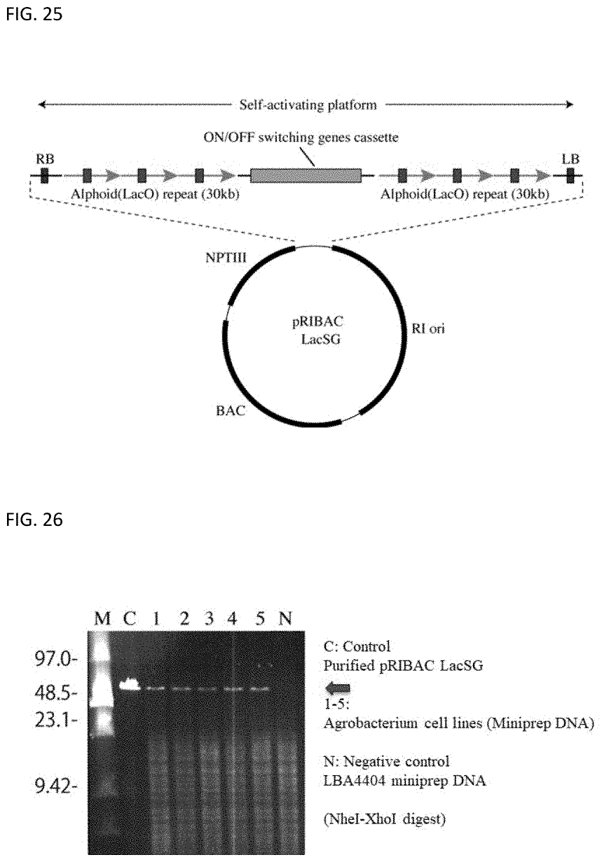

Finally, the LacO alphoid DNA fragment cut out from pRIBAC TW11.16 with NheI and NotI was inserted between the SpeI-NotI sites of the pRIBAC LW11.16 SG to complete a plasmid (pRIBAC LacSG) for producing a “self-activating type regulator expression platform”, a gene expression regulator vector (self-activating type) including a DNA sequence for expressing the gene expression regulator of the present invention (FIG. 25). In addition, a regulator expression construction (pRIBAC SG Alone: SEQ ID NO: 6) not including the LacO alphoid DNA sequence was also produced for comparison, and they were purified with a Large Construction kit (QIAGEN).

The pRIBAC LacSG plasmid produced using Escherichia coli and purified was introduced into electrocompetent cells of the Agrobacterium cell line LBA4404 (TAKARA). Subsequently, the cells were seeded on an LB plate containing kanamycin (30 μg/mL) to form single colonies. Multiple single colonies of Agrobacterium were picked up and cultured, and the plasmid DNA was collected therefrom by an alkali-SDS method and subjected to PFGE analysis (FIG. 26). A band with the same degree of migration as that of the introduced BAC plasmid was observed in a half or more of the analyzed strains to confirm that the repeat sequence of the LacO alphoid DNA was stably maintained. 1-(7) Acquisition of gene expression ON/OFF switching platform-introduced tobacco cultured cell BY-2 Culture of tobacco cultured cell BY-2

Nicotiana tabacum tobacco cultured cell BY-2 (RPC00001) provided from RIKEN, BioResource Research Center, Experimental Plant Division was used. The culture was carried out according to the protocol of RIKEN, the cell distributor, as follows. The medium used was a modified Linsmaier and Skoog (mLS) medium (Murashige and Skoog Plant Salt Mixture (FUJIFILM Wako Pure Chemical Corporation), 1 μg/mL thiamine hydrochloride, 0.1 mg/mL myo-inositol, 0.2 mg/mL KH2PO4, 30 mg/mL sucrose, 0.2 μg/mL 2,4-dichlorophenoxyacetic acid, pH 5.8). Shaking culture was performed at 130 rpm in a dark place at 28° C. On the 7th day after subculture, 1 mL of the culture solution was subcultured in 95 mL of the mLS medium.

Introduction of Gene Expression ON/OFF Switching Platform into Tobacco Cultured Cell BY-2

The gene expression ON/OFF switching platform (tetPF or LacPF) including the tetO alphoid DNA or the LacO alphoid DNA was inserted into a BY-2 chromosome by a method using Agrobacterium shown below. The Agrobacterium LBA4404 colony including the pRIBAC tetPF plasmid or the pRIBAC LacPF was inoculated in 10 mL of an LB medium containing antibiotics (50 μg/mL rifampicin, 25 μg/mL streptomycin, and 25 μg/mL kanamycin), followed by shaking culture at 120 rpm at 26° C. overnight. After washing with 1 mL of an LB medium three times, the cells were suspended in an LB medium so as to give an OD600 of 1.0. Five milliliters of the BY-2 cells in the logarithmic growth phase (cells 3 days after subculture) in the mLS medium were spread in a 10-cm dish, 100 μL of an Agrobacterium suspension was added thereto, and co-culture was performed for 2 days at 26° C. in the presence of 24 μM acetosyringone. The cells were collected and were washed with 10 mL of an mLS medium (containing 0.5 mg/mL Cefotax) four times. Selection was performed by culturing on an agar medium containing 0.5 mg/mL Cefotax and a selection drug (100 μg/mL kanamycin) at 28° C.

### EYFP Observation

EYFP fluorescence observation used a confocal laser scanning microscope LSM800 (Carl Zeiss AG) and a Plan-NEOFLUAR 5-times magnification lens or a Plan-NEOFLUAR 20-times magnification lens.

### GUS Staining

The BY-2 cells were suspended in a GUS staining solution (1 mM X-Gluc (5-bromo-4-chloro-3-indolyl-beta-D-glucuronide cyclohexylammonium salt), a 50 mM phosphate buffer pH 7.2, 0.5 mM K3[Fe(CN)₆], 0.5 mM K4[Fe(CN)₆], 0.1% Triton X-100), followed by incubation at 37° C. for several hours.

### Purification of Genomic DNA

About 45 mg of the cells were collected in a 2-mL tube for freeze-fracture (Yasui Kikai Corporation) and were frozen in liquid nitrogen. Metal cone was added thereto, and crushing was performed using Multi-beads shocker at 2800 rpm for 15 seconds. After addition of 260 μL of Cell Lysis buffer (Promega), 100 μL of Tail Lysis buffer (Promega), 20 μL of Proteinase K solution (Promega), and 20 μL of RNase A solution (Promega), the resulting suspension was centrifugated at room temperature at 14,000 rpm for 2 minutes. The supernatant was applied to the cassette of a Maxwell RSC Plant DNA kit (Promega) and was set to an automatic nucleic acid purification apparatus Maxwell 16 (Promega), and a plant DNA preparation protocol was implemented. The DNA was quantitatively measured using a Qubit dsDNA HS assay kit (Thermo Fisher Scientific).

qPCR

The purified genomic DNA was analyzed using a CFX96 real-time PCR system (Bio-Rad), SYBR Premix Ex TaqII (Takara Bio Inc.), and an alphoid DNA amplification primer set (Table 2). As the standard sample, purified tetPF or LacPF DNA was used. Normalization was performed using genomic DNA quantitative values.

The thus produced gene expression ON/OFF switching platform, a vector including a plant recombinant gene expression regulatory platform DNA sequence, was inserted in tobacco cultured cell BY-2 using Agrobacterium. As a result, 20 resistant calluses for tetPF and 8 resistant calluses for LacPF were formed. Furthermore, subculture was repeated using an agar medium containing 0.5 mg/mL Cefotax and a selection drug (100 μg/mL kanamycin) at 28° C. Ultimately, 16 calluses (strains) for tetPF and 3 calluses (strains) for LacPF could be stably cultured and maintained.

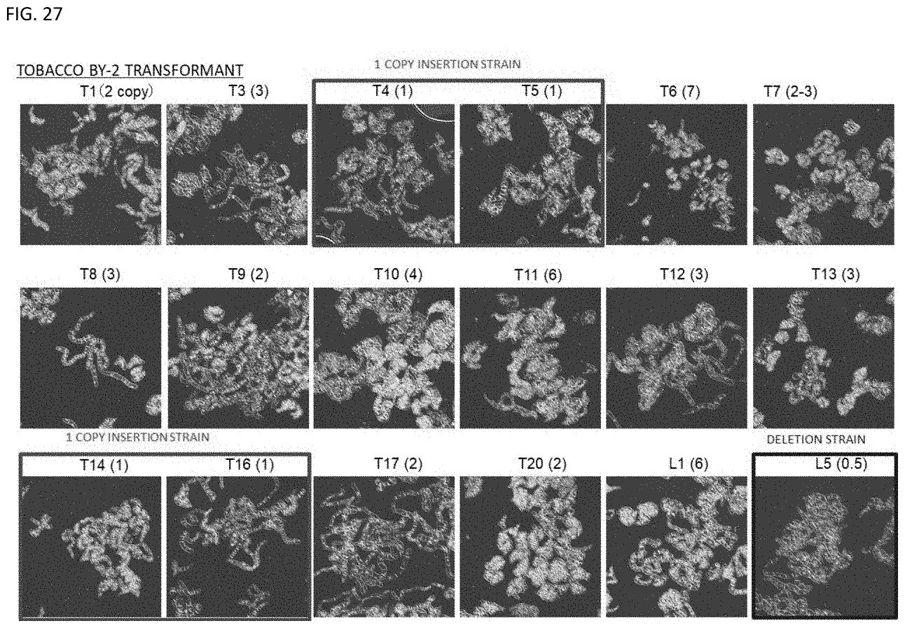

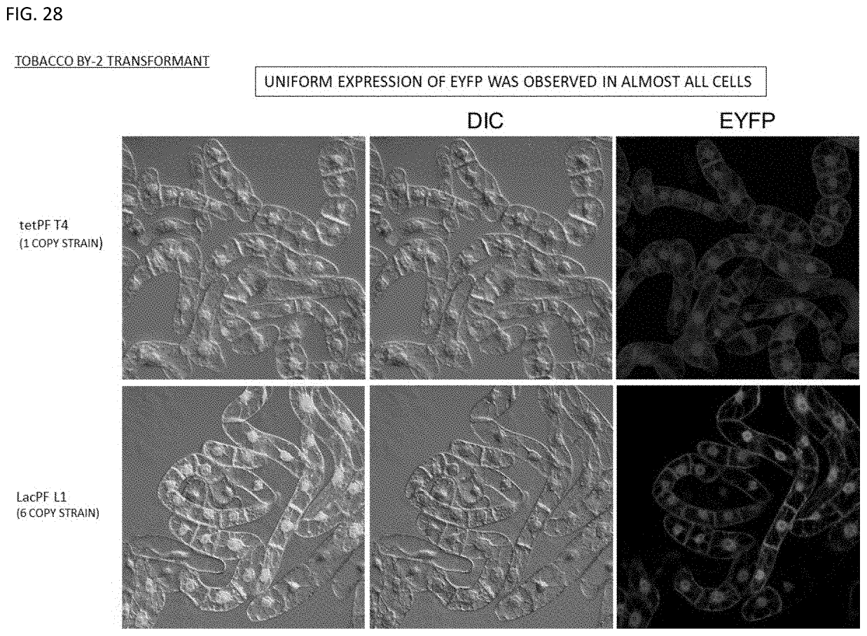

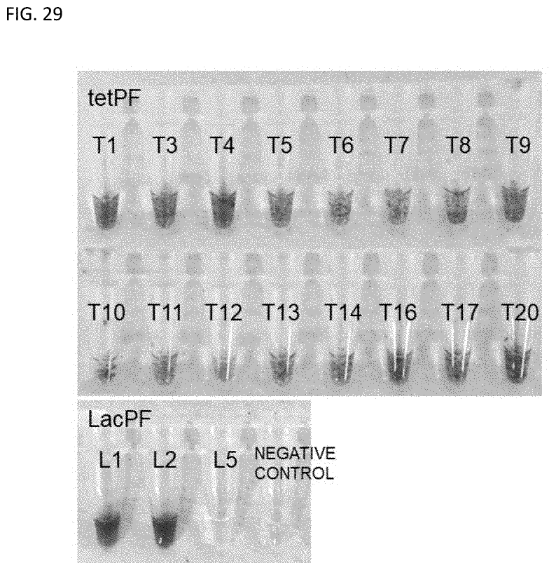

The gene expression ON/OFF switching platform is linked to three marker genes, NPTII (kanamycin resistant), EYFP (EYFP fluorescence), and GUS (β-glucuronidase) genes. Furthermore, an alphoid DNA (30 kb) including the tetO sequence or the LacO sequence is linked to both ends thereof. The obtained 16 tetPF strains and 3 LacPF strains were subjected to EYFP fluorescence observation and GUS staining in order to verify the insertion of the gene expression ON/OFF switching platform. As a result, expression of the EYFP gene and the GUS gene was confirmed in the strains excluding the LacPF strain L5 (FIGS. 27, 28, and 29).

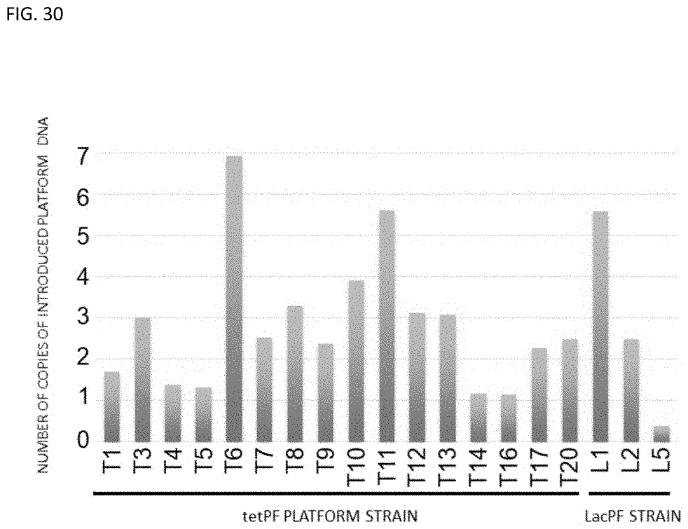

Furthermore, in order to verify the number of copies of the inserted gene expression ON/OFF switching platform, the genomic DNA was purified and subjected to qPCR. As a result, it was confirmed that four strains including the gene expression ON/OFF switching platform at one copy were also acquired (FIG. 30). In the LacPF L5 strain that was only strain not showing the EYPF fluorescence and the GUS activity, since the number of copies was presumed to be 0.5 or less, it was inferred that the T-DNA was inserted only from the left half of the alphoid DNA till NPTII (showing kanamycin resistance) into the genome (FIGS. 27, 28, and 29). In all strains, the EYPF fluorescence intensity, the GUS activity, and the NPTII resistance were consistent with the results of the measurement of the number of copies of the alphoid DNA to show high reliability of the results of the measurement of the number of copies.

### 1-(8) Analysis of Chromatin Structure by Chromatin Immunoprecipitation (ChIP) of BY-2 ON/OFF Switching Platform Candidate Strains

Crude Nuclear Fraction Preparation from BY-2 Cell and Crosslink of Chromatin

BY-2 (tetO platform candidate strain T4) cells cultured in a Murashige and Skoog medium (3% sucrose) were washed with an ice-cooled 50 mM potassium phosphate buffer (pH 5.8) of a volume equal to the cell volume twice and were suspended in the same buffer of a volume equal to the cell volume. Three milliliters of the suspension and 1 mL of ice-cooled 75% (w/v) glycerol were mixed, and the mixture was put in a tube dedicated for 50-mL Multi-beads shocker (Yasui Kikai Corporation) with a special metal cone and pre-cooled with ice and was crushed at 1,000 rpm for 1 minute five times (ice-cooling between each crushing). The cell homogenate was divided into four Eppendorf tubes, followed by centrifugation at 400×g (swing rotor) at 4° C. for 2 minutes. The precipitate containing nuclei was suspended in 1 mL of ice-cooled PBS, followed washing by similar centrifugation twice. The precipitate (about 0.3 mL×4 tubes) was suspended in PBS containing 2% formaldehyde in an amount of 3 times the amount of the precipitate, followed by a reaction at room temperature for 20 minutes to crosslink the chromatin. The crosslink reaction was stopped by mixing 60 μL of PBS containing 2.5 M glycine. Centrifugation was performed at 400×g (swing rotor) at 4° C. for 2 minutes, and the precipitate was washed with 1 mL of ice-cooled PBS by similar centrifugation twice. Furthermore, the precipitate was washed with 1 mL of ice-cooled TE (pH 8.0) by similar centrifugation and was stored at −80° C. after the supernatant was removed.

### Chromatin Immunoprecipitation

The precipitate (about 0.2 mL derived from about 0.4 g of cells) of the crude nuclear fraction stored at −80° C. was suspended in 0.5 mL of a sonication buffer (20 mM Tris-HCl buffer pH 8.0, 1 mM EDTA, 0.025% SDS, 0.5 mM DTT, 1.5 μM aprotinin, 20 μM leupeptin, 40 μM MG132), and nuclear destruction and chromatin cleavage were performed under appropriate conditions (for example, 15 cycles of a cycle consisting of ON for 30 sec/OFF for 30 sec) of Picoruptor (Diagenode). Centrifugation was performed at 4° C. ad 20,000×g (swing rotor) for 10 minutes, and the supernatant was collected as a chromatin fraction. The chromatin fraction was adjusted to a volume within a range of 2 to 5 times the fraction volume and a buffer composition of 0.3 M NaCl, 20 mM Tris-HCl pH 8.0, 0.5 mM EDTA, 5% glycerol, 0.05% SDS, 1% Triton X-100, 0.5 mM DTT, 1.5 μM aprotinin, 20 μM leupeptin, and 20 μM MG132, and Protein G magnetic beads (Dynabeads, Thermo Fisher Scientific) to which an antibody was previously bound was mixed therewith. As an input DNA, one-tenth amount of the chromatin fraction used in the immunoprecipitation reaction was maintained at 4° C. In the immunoprecipitation reaction of 0.5 mL, 1 μg of a monoclonal antibody and 5 μL of Protein G magnetic beads were used. The reaction was performed at 4° C. overnight while gently stirring with a rotator, and the beads were then collected with a magnet and washed with 0.3 mL of a washing buffer (0.55 M NaCl, 20 mM Tris-HCl pH 8.0, 1 mM EDTA, 5% glycerol, 0.1% SDS, 1% Triton X-100) three times. Subsequent treatment was also similarly performed for the input DNA. Magnetic beads were suspended in 80 μL of an elution buffer (0.15 M NaCl, 50 mM Tris-HCl pH 8.0, 10 mM EDTA, 0.5% SDS, 0.1 mg/mL RNase A) and were treated at 37° C. for 1 hour or more, and then 20 μL of 0.2 mg/mL Proteinase K was added thereto to digest proteins at 50° C. for 1 hour or more. Subsequently, the crosslink was removed by heating at 65° C. overnight. The beads were removed with a magnet, and DNA was purified from 0.1 mL of the resulting solution using MinElute PCR Purification Kit (Qiagen N.V.) and was eluted with 50 μL of TE. Real time PCR using a SYBR Green fluorescence dye was performed using 2.5 μL of the eluted DNA as a template.

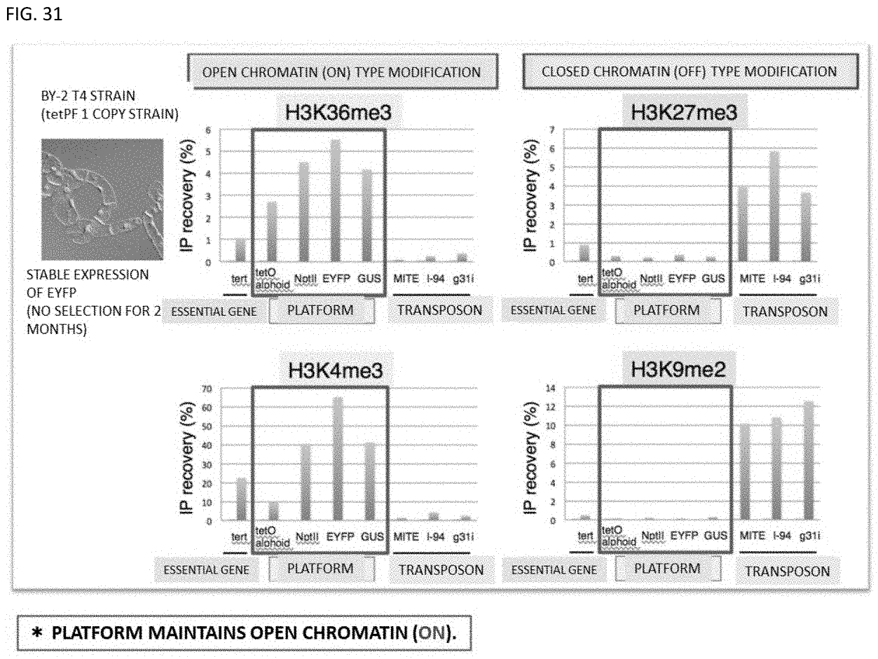

BY-2 tet platform candidate strains were cultured for 2 months in a Murashige and Skoog medium not containing kanamycin, a selection pressure against inserted gene expression, and the insertion regions of the synthetic repetitive DNA (tetO alphoid), the kanamycin resistance gene (NPTII), the fluorescence marker gene (EYFP), and the enzyme marker gene (GUS), the telomerase gene (TERT) as the expression gene on the genome, and the histone modifications of three transposons (MITE, 1-94, and g31i) as the heterochromatinized regions were evaluated by chromatin immunoprecipitation (ChIP) (FIG. 31, Table 2). In all of the platform regions, the levels of open chromatin type modifications, trimethylation of the 36th lysine of histone H3 (H3K36me3) and trimethylation of the 4th lysine of histone H3 (H3K4me3) were both higher than that of the essential gene TERT. On the other hand, in the transposon region, which is a repression type chromatin, the H3K36me3 and H3K4me3 modifications were not substantially detected, but closed chromatin type modifications, trimethylation of the 27th lysine (H3K27me3) of histone H3 and dimethylation of the 9th lysine of histone H3 (H3K9me2) were detected at very high levels. In contrast, in the platform region, the repression type modifications, H3K27me3 and H3K9me2, were not substantially detected. These analytical results revealed that the platform can maintain the open chromatin structure under no induction conditions for a long time of no selection pressure.

### Antibody Used in Immunoprecipitation

Normal mouse IgG (SantaCruz SC2025)

Anti-H3K9me2 (Monoclonal Antibody Laboratories MABI0307)

Anti-H3K27me3 (Monoclonal Antibody Laboratories 1E7)

Anti-H3K36me3 (Monoclonal Antibody Laboratories MA333B)

Anti-H3K4me3 (Monoclonal Antibody Laboratories MA304B)

TABLE 2
[Primer set used in PCR]

| Target | Sequence 1 (5′→3′) | Sequence 2 (5′→3′) |
|---|---|---|
| TERT | AGAGAGGTTGGGTTCATC | TGAGATCATCCAGCACACTCA |
| | TGT | (SEQ ID NO: 23) |
| | (SEQ ID NO: 22) | |
| Alphoid | GGGATCACTAGCAATAAA | TCCTTCTGTCTCGTTTTTATG |
| | AGGTAGAC | GC |
| | (SEQ ID NO: 24) | (SEQ ID NO: 25) |
| NPTII | GCGCCCGGTTCTTTTTGT | TTCCCGCTTCAGTGACAACGT |
| | CAA | (SEQ ID NO: 27) |
| | (SEQ ID NO: 26) | |
| EYFP | AGATCCGCCACMCATCGA | TCGTTGGGGTCTTTGCTCAGG |
| | GG | (SEQ ID NO: 29) |
| | (SEQ ID NO: 28) | |
| GUS | CGACGCTCACACCGATAC | CTCTGCCGTTTCCAAATCGCC |
| | CAT | (SEQ ID NO: 31) |
| | (SEQ ID NO: 30) | |
| MITE | TCACGAGGACTAGGTACC | TACGCCATGAATCTCGACCA |
| | GA | (SEQ ID NO: 33) |
| | (SEQ ID NO: 32) | |
| 1-94 | ACCTTGTTGACTTGGTTT | TGTTGGTGTGAAGAAATGAGA |
| | GGT | GT |
| | (SEQ ID NO: 34) | (SEQ ID NO: 35) |
| g31i | TCGTTCGGAGGTGATTTG | CCCGAGACCTCAACCAAACA |
| | GT | (SEQ ID NO: 37) |
| | (SEQ ID NO: 36) | |

[PCR conditions]
2 × SYBR premix ExTaq (Takara) 5 μL
50 μM primer 1 0.1 μL
50 μM primer 2 0.1 μL
Template DNA 2.5 μL
H₂O to final to final 10 μL

After 95° C. for 60 sec, 40 cycles of the following 3 steps: 95° C. 10 sec; 62° C. 30 sec; 72° C. 60 sec
A standard curve was prepared from a dilution series of input DNA, and the relative amount of the target in immunoprecipitated DNA to the input DNA was calculated.

### 1-(9) Effect of ON/OFF Regulator on Gene Expression ON/OFF Switching Platform

Introduction ON/OFF Regulator Expression DNA Sequence into Gene Expression ON/OFF Switching Platform One Copy Strain

A colony of Agrobacterium LBA4404 including an ON/OFF regulator expression cassette that is the DNA sequence for expressing the gene expression regulator of the present invention was inoculated in 10 mL of an LB medium containing antibiotics (50 μg/mL rifampicin, 25 μg/mL streptomycin, and 25 μg/mL kanamycin), followed by shaking culture at 120 rpm at 26° C. overnight. After washing with 1 mL of an LB medium three times, the cells were suspended in an LB medium so as to give an OD600 of 1.0. Five milliliters of the BY-2 cells in the logarithmic growth phase (cells 3 days after subculture) in the mLS medium were spread in a 10-cm dish, 100 μL of an Agrobacterium suspension was added thereto, and co-culture was performed for 3 days at 26° C. in the presence of 24 μM acetosyringone. The cells were collected and were washed with 10 mL of an mLS medium (containing 0.5 mg/mL Cefotax) four times. Selection was performed by culturing on an agar medium containing 0.5 mg/mL Cefotax and a selection drug (50 μg/mL hygromycin) at 28° C.

### EYFP Observation

EYFP fluorescence was observed using a penlight for EYFP observation, Handy Green Pro Plus for YFP (RelyOn Ltd.)

TABLE 3
Primer set used in PCR
Target Sequence 1 (5′→3′)
Alphoid GGGATCACTAGCAATAAAAGGTAGAC (SEQ ID NO: 24)
Sequence 2 (5′→3′)
TCCTTCTGTCTCGTTTTTATGGC (SEQ ID NO: 25)
PCR conditions:
2 × SYBR premix ExTaq (Takara) 5 μL
50 μM primer 1 0.1 μL
50 μM primer 2 0.1 μL
Template DNA 2.5 μL
H₂O to final 10 μL

After 95° C. for 60 sec, 40 cycles of the following 3 steps:

### 95° C. 10 sec

### 62° C. 30 sec

### 72° C. 60 sec

The effect of the ON/OFF regulator (gene expression regulator) was verified. TetR-SUV39H1MTD and tetR-tTS were used as factors on the OFF-side, and rtetR-KAT7HD was used as a factor on the ON-side. Each of the expression cassettes of these genes was individually cloned on an appropriate Agrobacterium vector containing a hygromycin resistance gene. Then, as strains each expressing the EYFP gene and the GUS gene and including the gene expression ON/OFF switching platform at one copy, tetPF T4 and T5 were selected, and the expression cassettes of the OFF-side repressor (tetR-SUV39H1MTD or tetR-tTS) and the ON-side regulator (rtetR-KAT7HD) included in the Agrobacterium vector were individually introduced into the strains by the Agrobacterium method.

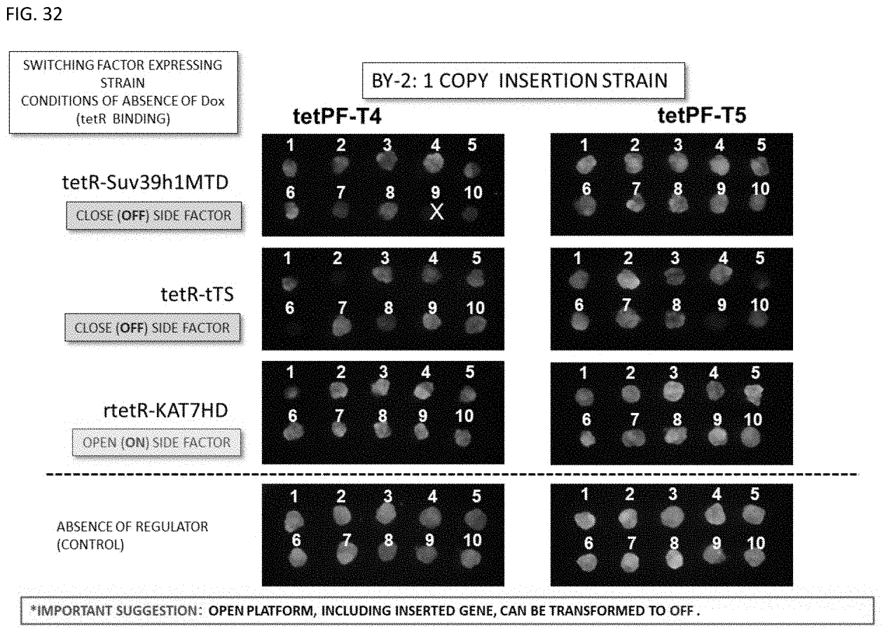

As a result, in both the tetPF T4 strain and T5 strain, about 200 resistant calluses occurred for each regulator. The selection medium did not contain tetracycline-type antibiotics, and it was expected that tetR-SUV39H1MTD or tetR-tTS bound to the ON/OFF platform to express the OFF-side effect (Table 1). In order to verify the possibility, 10 calluses were randomly selected for each factor, the strains were subcultured for about one month in the condition without selection of the gene expression ON/OFF platform and with selection of the regulator (agar medium containing no kanamycin and containing hygromycin), and EYFP fluorescence was observed. As a result, in tetR-SUV39H1MTD and tetR-tTS, although there were differences in response by the strains, multiple calluses with notably decreased fluorescence of EYFP were observed (FIG. 32). This demonstrates that the platform was changed from ON to OFF as expected. These OFF-side repressors can bind to the alphoid sequence only. Accordingly, the fact that multiple calluses repressed in the EYFP expression of the gene region inside the platform were obtained strongly suggests a possibility that the whole gene expression ON/OFF platform was switched to the OFF-side chromatin structure as expected. In contrast, in the calluses into which the ON-side regulator rtetR-KAT7HD was introduced, although EYFP expression was detected, fluctuations in the level were observed. In the callus into which no regulator was introduced, such a tendency was not observed.

1-(10) Acquisition of Gene Expression ON/OFF Switching Platform-Introduced Arabidopsis thaliana
Introduction of Gene Expression ON/OFF Switching Platform into Arabidopsis thaliana

The gene expression ON/OFF switching platform (tetPF) containing the tetO alphoid DNA was inserted into the Arabidopsis thaliana chromosome by a method using Agrobacterium shown below. A colony of Agrobacterium GV3101 including a pRIBAC tetPF plasmid was inoculated in 20 mL of an LB medium containing antibiotics (50 μg/mL rifampicin, 25 μg/mL gentamicin, and 25 μg/mL kanamycin), followed by culture at 120 rpm at 25° C. overnight. Five milliliters of the Agrobacterium culture solution was added to 150 mL of an LB medium containing antibiotics (50 μg/mL rifampicin, 25 μg/mL gentamicin, and 25 μg/mL kanamycin), followed by further culture at 120 rpm at 25° C. overnight. After centrifugation of 150 mL of the Agrobacterium culture solution and removal of the supernatant, Agrobacterium was suspended in 30 mL of an infiltration buffer (½ concentration of Murashige and Skoog Plant Salt Mixture (FUJIFILM Wako Pure Chemical Corporation), 5% sucrose, Gamborg's vitamin solution (Sigma), 0.01 μg/mL 6-benzylaminopurine, 0.02% Silwet L77). Arabidopsis thaliana seeded and cultivated at a 16-hour light and 8-hour dark cycle at 22° C. for about 2 months was immersed in the suspension for 30 seconds. Arabidopsis thaliana was wrapped in cellophane and laid down horizontally and was left in dark overnight. The cellophane was removed, and the Arabidopsis thaliana was cultivated at a 16-hour light and 8-hour dark cycle at 22° C. for seed growing. The seeds were seeded on an agar medium (Murashige and Skoog Plant Salt Mixture (FUJIFILM Wako Pure Chemical Corporation), 3% sucrose, 2.3 mM MES, pH 5.7, Gamborg's Vitamin Solution (Sigma), 0.8% agar) containing 0.5 mg/mL Cefotax and a selection drug (30 μg/mL kanamycin) to perform selection. EYFP observation

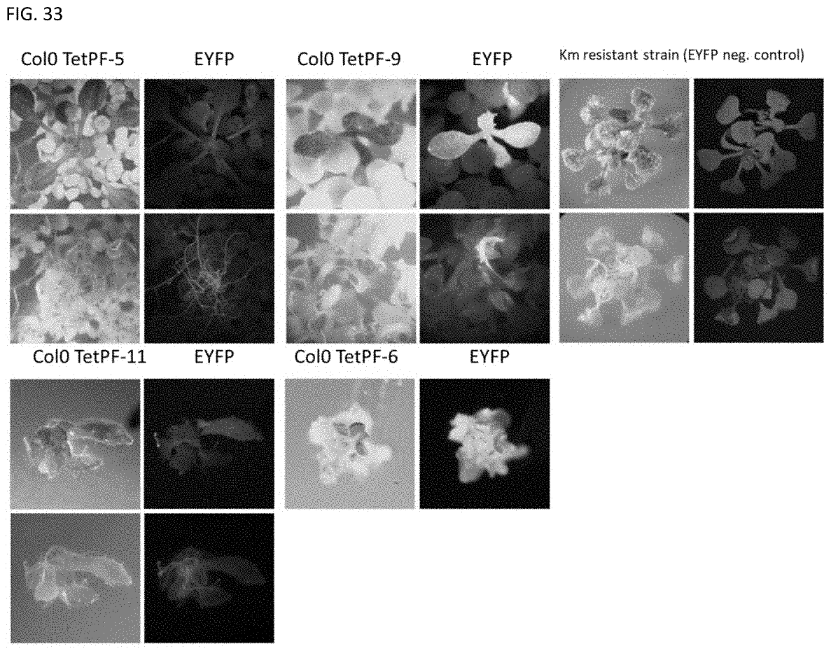

EYFP fluorescence was observed with a stereo microscope equipped with an EYFP observation module (Leica Microscopes). The gene expression ON/OFF switching platform was introduced into Arabidopsis thaliana using Agrobacterium. Selection was performed for about 40,000 seeds, and 9 seeds of kanamycin resistant strains were obtained. Furthermore, in order to verify the insertion of the gene expression ON/OFF switching platform, expression of the marker gene EYFP was verified by observation of EYFP fluorescence. As a result, in all of them, EYFP fluorescence was observed (FIG. 33). In six strains, EYFP fluorescence was mainly observed in the roots. In one strain, strong EYFP fluorescence was observed in the whole plant body. In this strain, there was a possibility that the platform was inserted in a region that was easily expressed or a plurality of copies were inserted. In two strains, although EYFP fluorescence was observed in roots and so on, abnormal morphology was observed. It is inferred that the platform was inserted in a region that adversely affects development and growth. These results revealed that a large number of strains in which a gene expression ON/OFF switching platform is at least inserted into the genome of Arabidopsis thaliana can be obtained and that expression of the EYFP gene of the platform is also maintained as in the tobacco BY-2 cell.

1-(11) Improvement by Construction of Fusion Binary Vector pRIBAC LacSG-tetPF

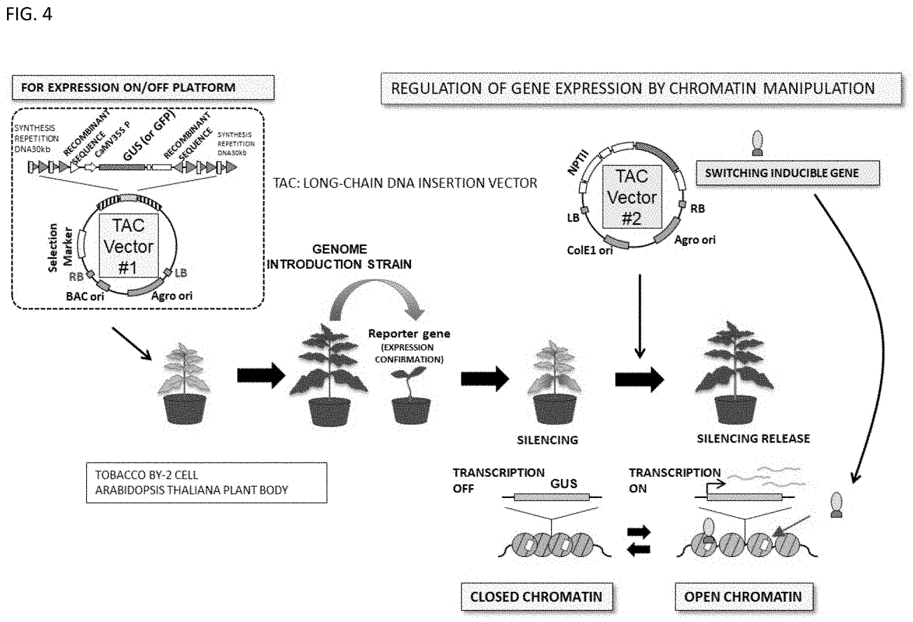

In order to regulate the gene expression on a multigene (a plurality of genes) expression cassette sequence (genome recombination station) included in the expression ON/OFF switching platform, it is necessary to infect an expression ON/OFF switching platform (plant recombinant gene expression regulatory platform) insertion strain (obtained by infection with a pRIBAC tetPF vector via Agrobacterium) with a vector (gene expression regulator vector), such as pRIBAC LacSG, again via Agrobacterium to express the regulator from another insertion site. Accordingly, the expression ON/OFF switching requires two-step gene introduction (FIGS. 4 and 6).

In addition, when a gene on the genome recombination station is harmful to growth of plants, it is necessary to obtain a transformed plant while repressing the gene expression on the genome recombination station. In this case, there is a problem that expression of the drug-selection marker present in the genome recombination station is also repressed and the drug-selection marker cannot be used for selection.

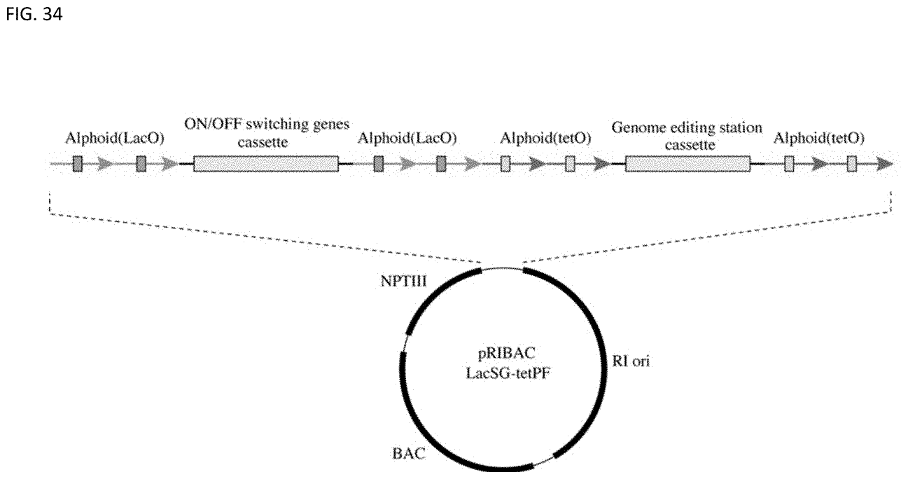

In order to overcome these problems, a construction, pRIBAC LacSG-tetPF, consisting of the self-activating-type platform of pRIBAC LacSG (FIG. 25) and the expression ON/OFF switching platform of pRIBAC tetPF (genome recombination station: FIG. 16) linked to each other on one vector was produced (FIG. 34). The linking was performed by a method of inserting a tetPF fragment cut out with NheI-NotI into the SpeI-NotI site of LacSG.

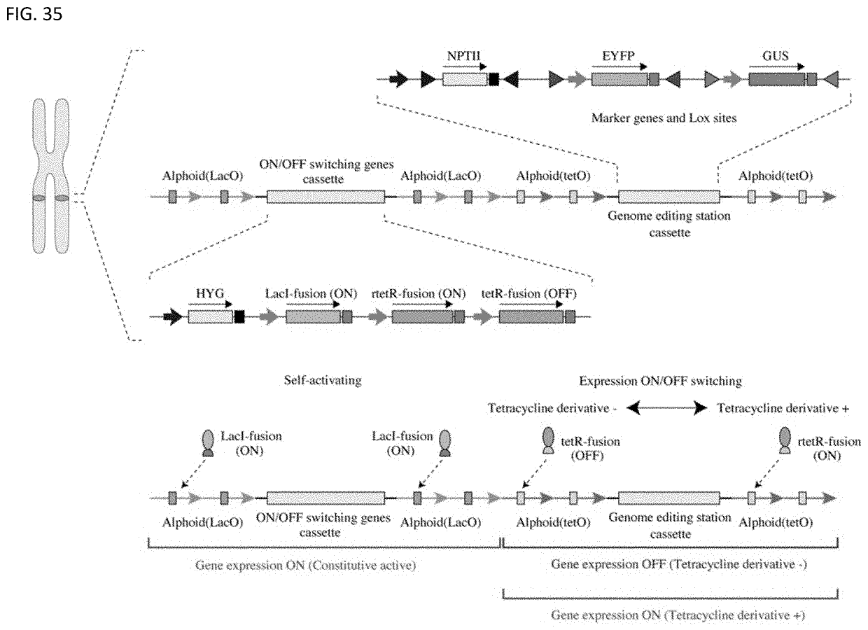

An advantage of this vector is that an ON/OFF switching platform (genome recombination station) insertion strain that can regulate expression induction can be obtained by one-time infection with Agrobacterium. In addition, another advantage is that gene expression on the genome recombination station can be repressed from the beginning of Agrobacterium infection by binding the repressor (tetR-fusion (OFF)) to be expressed from the LacSG side to the tetO alphoid DNA side (FIG. 35, tetracycline derivative: −).

Furthermore, since the LacSG is a self-activating-type platform, even if the adjacent chromatin structure on the tetPF side is repressive to the gene expression, it is expected that the LacSG side can express a drug resistance gene and a regulator independent from the tetPF side by binding a gene expression activator (LacI-fusion (ON)) to the LacO alphoid DNA region. Consequently, for example, it is expected that a transformant strain can be obtained while keeping the tetPF side OFF by selection using a Hyg resistance gene present on the LacSG side (FIG. 35: tetracycline derivative: −).

Expression of a gene on the genome recombination station can also be induced by converting the binding from tetR-fusion (OFF) to rtetR-fusion (ON) through addition of a tetracycline derivative at necessary timing (FIG. 35, tetracycline derivative: +).

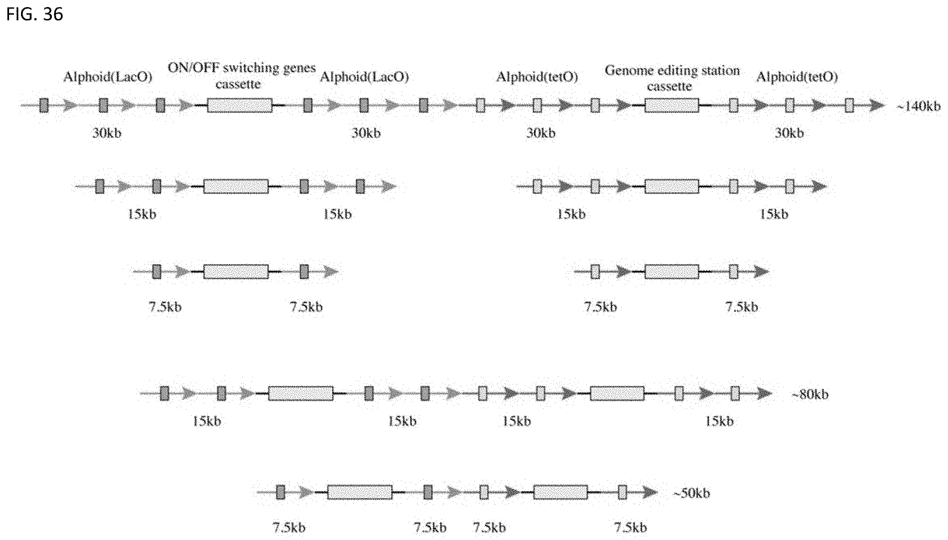

Incidentally, the size of this fusion vector, pRIBAC LacSG-tetPF, is close to 140 kb with the T-DNA insert alone, and cloning in Escherichia coli using BAC is expected to become more difficult. In addition, when a plant is infected with the T-DNA of this size via Agrobacterium, a decrease in the efficiency of infection is also expected. Accordingly, it is preferable to miniaturize the lengths of the tetO and LacO alphoid DNAs in the pRIBAC LacSG-tetPF to ½ and ¼ (FIG. 36).

2. Insertion of Gene into Multigene (a Plurality of Genes) Expression Cassette Sequence in Plant Recombinant Gene Expression Regulatory Platform DNA Sequence

### 2-(1) Incorporation of Genome Recombination Station on Expression ON/OFF Switching Platform

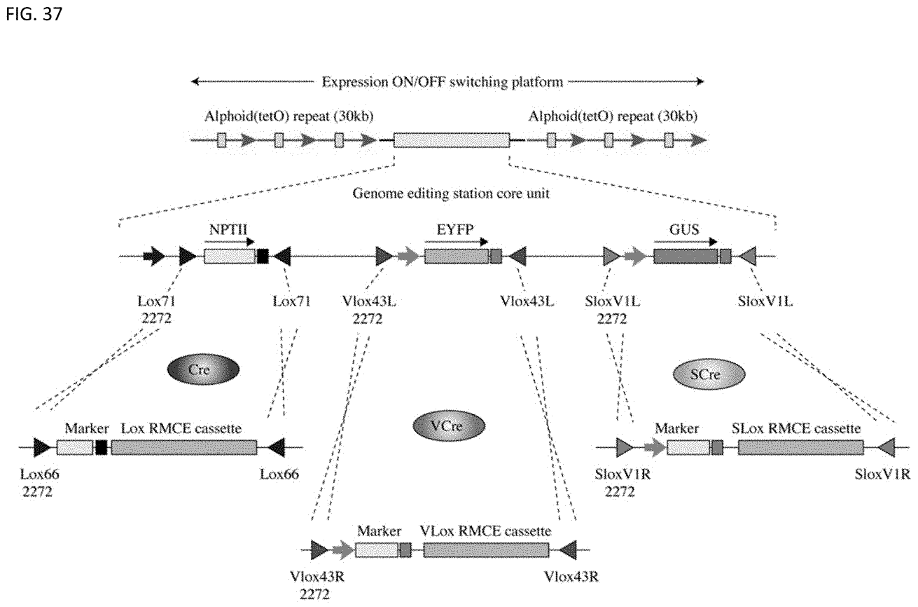

A system that can independently exchange each of the marker gene cassettes included in the multigene (a plurality of genes) expression cassette sequence of the expression ON/OFF switching platform for another arbitrary gene or sequence by a cassette exchange reaction was constructed by appropriately arranging a site-specific recombinant enzyme site (or a mutant site thereof), such as LoxP, beside the marker gene on the expression ON/OFF switching platform. This system is summarized in FIG. 37.

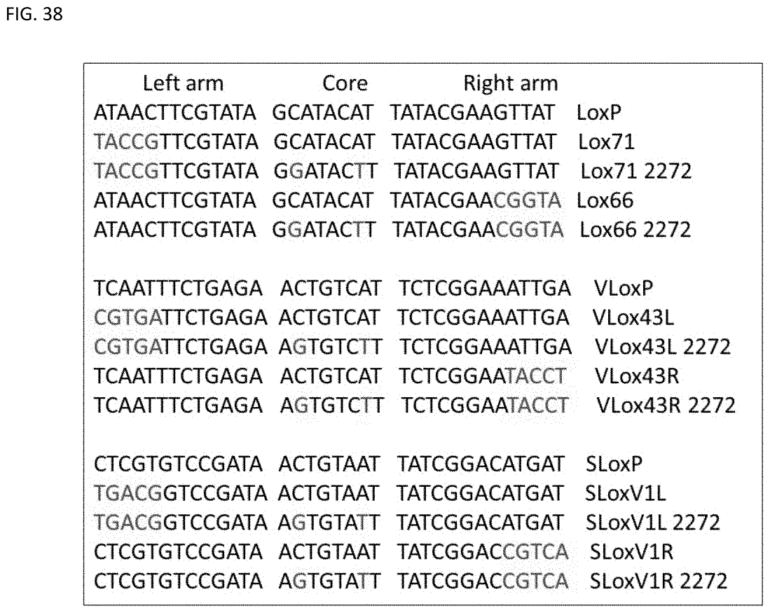

Among the three marker gene cassettes (NPTII, EYFP, and GUS) on the expression cassette sequence, in the NPTII gene cassette, which is a drug-selection marker, the Lox71 2272 site was inserted between the NOS promoter and the NPTII gene, and the Lox71 site was inserted in the downstream of the NOS terminator in the opposite direction to the Lox71 2272 site. Although the Cre protein is an enzyme recombines between two sites based on the LoxP sequence, since the Lox71 2272 site and the Lox71 site differ in the sequence of the core region site in the Lox sequence, recombination does not occur between these two sites (FIG. 38, Lee, G. and Saito, I., Role of nucleotide sequences of loxP spacer region in Cre-mediated recombination, Gene, 1998, Aug. 17, 216(1): 55-65). In contrast, the Lox66 2272 and the Lox66 having the same core sequence can recombined with the Lox71 2272 site and the Lox71 site, respectively. In addition, since the Lox66 2272 and the Lox71 2272 site have mutations in the sequences of the right arm and left arm sites, respectively, recombination between them causes a Lox 2272 site having mutations on both arms on the genome recombination station. In this case, it is known that in the site having mutations on both arms, the efficiency of recombination with another Lox site is significantly decreased. In addition, the same is feasible between the Lox71 site and the Lox66 site. Accordingly, when the cassette between the Lox66 2272 site and the Lox66 site is incorporated between the Lox71 2272 site and the Lox71 site of the genome recombination station by Cre, in the recombined cassette, the frequency of recombination by the Cre protein is notably decreased, and it is expected that the cassette is stably incorporated on the genome (FIGS. 37 and 38).

The VLox43L 2272 site and the VLox43L site are inserted in the upstream of the promotor of the EYFP gene cassette and the downstream of the terminator, respectively, in the opposite direction. Although a general VLox site is recombined by a CRE-like recombinant enzyme, VCRE enzyme, derived from vibrio, since the VLox43L 2272 site and the VLox43L site on the genome recombination station differ in the sequence of the core region site, recombination does not occur (Suzuki, E. and Nakayama, M., VCre/VloxP and SCre/SloxP: new site-specific recombination systems for genome engineering, Nucleic Acids Res., 2011, April, 39(8): e49). An arbitrary sequence can be inserted into the genome recombination station by introducing the sequence sandwiched by the VLox43R 2272 site and the VLox43R site thereto from the outside and performing recombination using VCRE. In addition, since the VLox43L sequence and the VLox43R sequence have mutations in the sequences of the left arm site and the right arm site, respectively, as in the Lox66 and the Lox71 site, in the sequence inserted once on the genome recombination station by recombination, the frequency of recombination by the VCRE protein is notably decreased, and it is expected that the sequence is stably incorporated on the genome (FIGS. 37 and 38).

The SLoxV1L 2272 site and the SLoxV1L site are inserted in the upstream of the promotor of the GUS gene cassette and the downstream of the terminator, respectively, in the opposite direction. Although a general SLox site is recombined by a CRE-like recombinant enzyme, SCRE enzyme, derived from Shewanella, since the SLoxV1L 2272 site and the SLoxV1L site on the genome recombination station differ in the sequence of the core region site, recombination does not occur (Suzuki and Nakayama, 2011). An arbitrary sequence can be inserted into the genome recombination station by introducing the sequence sandwiched by the SLoxV1R 2272 site and the SLoxV1R site thereto from the outside and performing recombination using VCRE. In addition, since the SLoxV1L sequence and the SLoxV1R sequence have mutations in the sequences of the left arm site and the right arm site, respectively, as in the Lox66 and the Lox71 site, in the sequence inserted once on the genome recombination station by recombination, the frequency of recombination by the VCRE protein is notably decreased, and it is expected that the sequence is stably incorporated on the genome (FIGS. 37 and 38).

Incidentally, these CRE, VCRE, and SCRE recombinant enzymes specifically act on corresponding Lox, VLox, and SLox sites, respectively, and do not cross-react with other sites. For example, VCRE performs recombination between the VLox sequences and does not perform recombination at the Lox and SLox sites.

In insertion of these various Lox sequences, PCR primers produced by oligo-DNA synthesis were used. These PCR primers were added with not only the respective corresponding insertion sites and sequences to be annealed but also various Lox sequences (Lox71 2272, Lox71, VLox43L 2272, VLox43L, SLoxV1L 2272, and SLoxV1L). By using these primers, constructions were produced by combining each component of the gene cassette present on the expression ON/OFF switching platform and various Lox sequences. The respective gene cassettes or the tetO alphoid DNA sequence all have the NheI, SpeI, and NotI sites, and can link arbitrary sequence in the forward direction as many times as required using the method of FIG. 11. The construction of FIG. 37 was produced by this linking method using Escherichia coli strain DH10B (see FIGS. 11, 12, and 14 to 18). It was also confirmed that the produced plasmid is stably maintained in Agrobacterium cells (FIG. 18). Additional Example: 1-(7) Acquisition of gene expression ON/OFF switching platform-introduced tobacco cultured cell BY-2

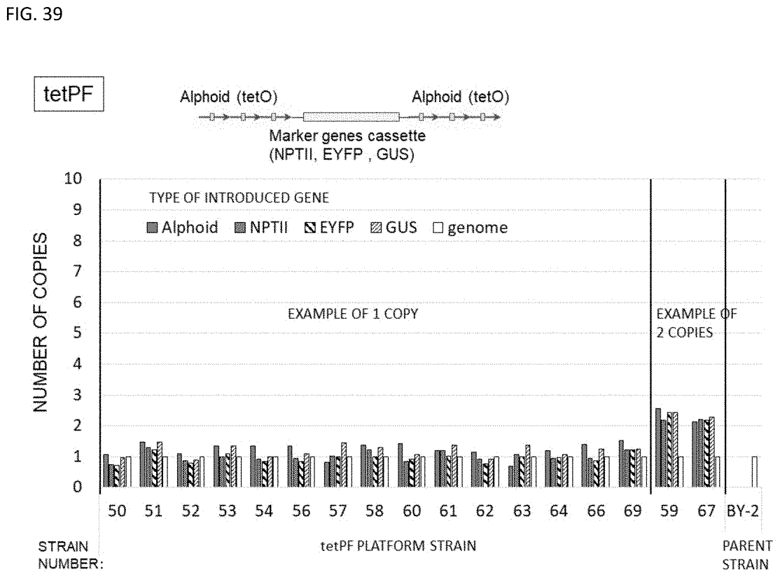

Furthermore, the gene expression ON/OFF switching platform was introduced into the tobacco cultured cell BY-2 using the method described in Example 1-(7) to newly obtain a 214 strain that can stably culture and maintain tetPF. In order to select a strain including the gene expression ON/OFF switching platform at one copy, the genomic DNA was purified and subjected to qPCR. In order to investigate the number of copies in detail, PCR primers for all of the three marker genes (NPTII, EYFP, and GUS) and the alphoid DNA contained in the gene expression ON/OFF switching platform were used (Table 2). As a result, 74 strains were obtained as one copy candidate strains (FIG. 39 shows 15 examples of strains of one copy and 2 examples of strains of two copies).

### Effect on the Number of Gene Introduction Copies by Gene Expression ON/OFF Switching Platform

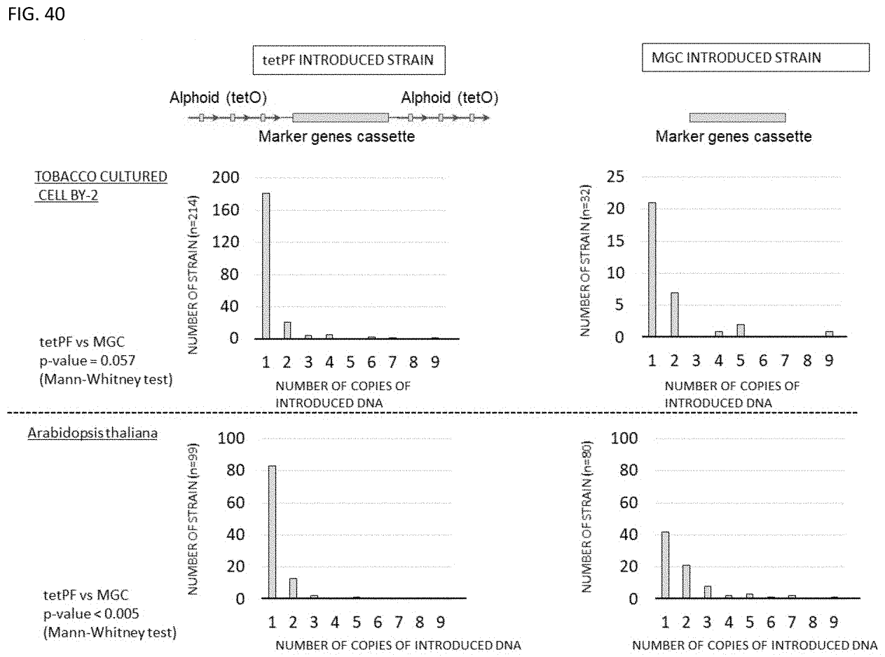

The number of copies of the 214 strains thus obtained by introduction of the gene expression ON/OFF switching platform tetPF into the tobacco cultured cell BY-2 was analyzed, and as a result, an effect of increasing the probability of introducing a target gene with a low number of copies was obtained by using the gene expression ON/OFF switching platform. The technology that can obtain a one-copy strain with high probability has high industrial usefulness. Accordingly, a construction MGC consisting of only the gene expression ON/OFF switching platform tetPF and a marker gene cassette not containing the alphoid DNA (FIG. 40) was similarly introduced into the tobacco cultured cell BY-2 using Agrobacterium, and the number of copies of the introduced DNA was relatively analyzed by qPCR. When the tobacco cultured cell BY-2 includes the alphoid DNA platform, the rate of introduction at one copy tended to be high. The same experiment was also performed using Arabidopsis thaliana by the method described in Example 1-(10), and as a result, when the alphoid DNA platform is included, the rate of introduction at one copy tended to be high, and in a Mann-Whitney test, and a particularly high significant difference was shown in Arabidopsis thaliana. These results demonstrated that the gene introduction using the gene expression ON/OFF switching platform has an effect of decreasing the number of copies.

### Additional Example: 1-(9) Effect of ON/OFF Regulator on Gene Expression ON/OFF Switching Platform

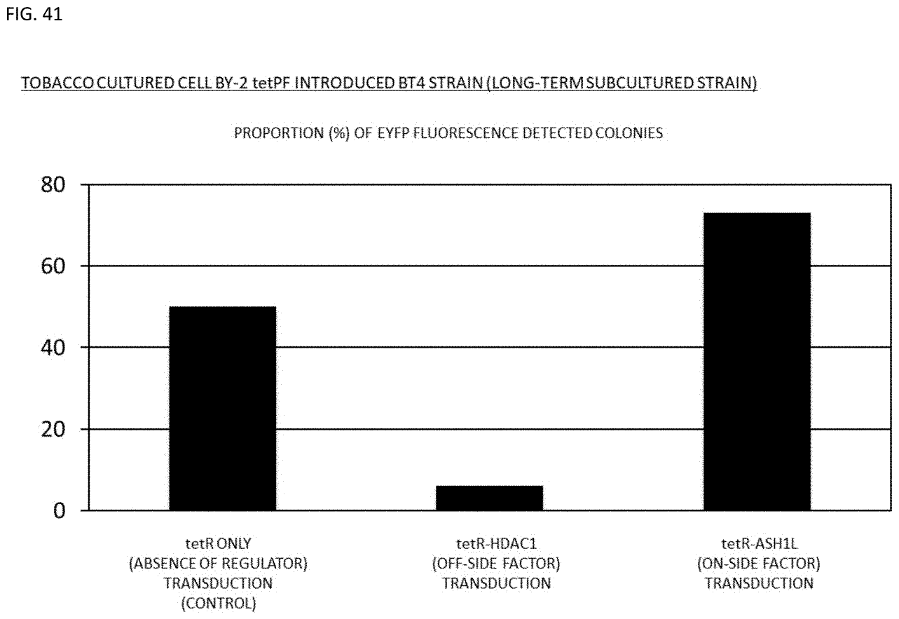

The tetPF T4 strain containing the gene expression ON/OFF switching platform at one copy described in Example 1-(9) was subcultured for a long period (about 1.5 years) to obtain a long-term cultured tetPF BT4 strain including cells (about 50%) repressed in the expression of the epigenetically introduced gene (EYFP) on the platform. In order to verify the ON/OFF switching effect, an experiment for verifying the effect of the ON/OFF regulator was performed this long-term cultured tetPF BT4 strain (FIG. 41).

TetR-HDAC1 was used as a factor on the OFF-side, tetR-ASH1L was used as a factor on the On-side, and tetR fused with no regulator was used as a control. Each of the expression cassettes of these genes was individually cloned on an appropriate Agrobacterium vector containing a hygromycin resistance gene. The vectors were transduced into the long-term cultured tetPF BT4 strain by the Agrobacterium method using the method described in Example 1-(9). The transduced cells were allowed to form colonies on a hygromycin-containing solid medium, and the numbers of colonies showing EYFP fluorescence and colonies not showing EYFP fluorescence were counted (FIG. 41). The bar graph of FIG. 41 shows the rate of colonies showing EYFP fluorescence in the whole colonies. As a result, when the OFF-side repressor (tetR-HDAC1) was introduced, the rate of colonies showing EYFP fluorescence was decreased; and when the ON-side regulator (tetR-ASH1L) was introduced, the rate of the colonies showing EYFP fluorescence was increased. The results above demonstrate that the expression of the epigenetically repressed gene on the ON/OFF platform is restored by binding of the ON-side regulator to the platform, the expression is repressed by binding of the OFF-side repressor, and ON/OFF switching of the whole area of the platform is possible.

Additional Example: 1-(10) Acquisition of Gene Expression ON/OFF Switching Platform-Introduced Arabidopsis thaliana

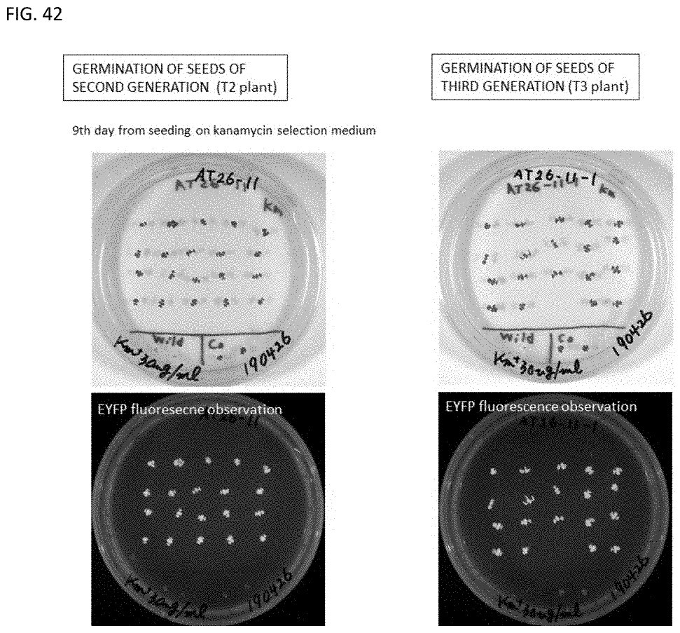

Seeds of the next generation and the next next generation of the gene expression ON/OFF switching platform one-copy-introduced candidate strains were acquired, and the characteristics of kanamycin resistant and EYFP fluorescence were verified (FIG. 42). The results demonstrate that the gene expression ON/OFF switching platform can be stably maintained even if the generation advances.

### Acquisition of Gene Expression ON/OFF Switching Platform-Introduced Benthamiana Tobacco

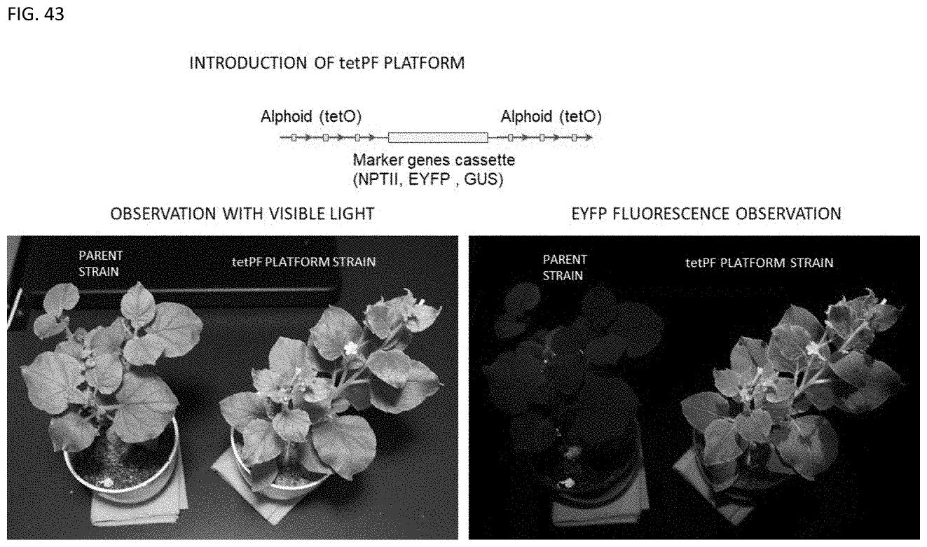

In addition to Arabidopsis thaliana of the family Cruciferae, in also tobacco of the family of Solanaceae, a gene expression ON/OFF switching platform-introduced strain was acquired. A gene expression ON/OFF switching platform (tetPF) including tetO alphoid was inserted into a benthamiana tobacco chromosome using Agrobacterium. A colony of Agrobacterium GV3101 or LBA4404 including a pRIBAC tetPF plasmid was inoculated in an LB medium containing antibiotics and was subjected to shaking culture overnight so as to give an OD600 of 0.5 to 1.0. Leaves of benthamiana tobacco grown in a Murashige and Skoog (MS) medium containing Gamborg's B5 vitamins (MSB5 medium) were cut into discs and were immersed in an Agrobacterium solution for 10 minutes. The disc-shaped leaves were placed on filter paper put on a co-culture medium and co-cultured for 3 days. The leaves were transferred to an MSB5 medium containing a selection drug to induce callus formation and chute formation. The chute was transferred to an MSB5 medium to form a root. The differentiated plant body (T1 generation) was transferred to soil and was allowed to grow to acquire the next generation (T2 generation). The T1 generation was confirmed that the gene expression ON/OFF switching platform was inserted by observation of the EYFP fluorescence of the marker gene (FIG. 43). So far, 40 or more strains of the T1 generation were acquired, and 20 or more strains of the T2 generation were acquired. Since EYFP fluorescence was observed in also the T2 generation, it is demonstrated that the gene expression ON/OFF switching platform is maintained beyond generations.

Additional Example: 1-(11) Improvement by Construction of Linked Binary Vector pRIBAC LacSG-tetPF

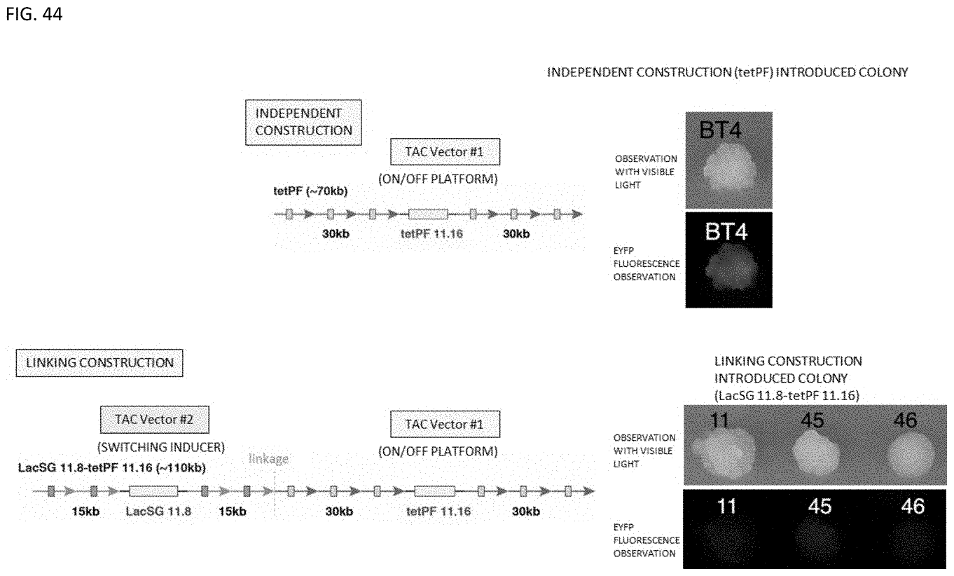

Whether a transformant strain can be acquired while keeping the tetPF side OFF or not was investigated by selection using the Hyg resistance gene present on the LacSG side of the linked binary vector shown in FIG. 35. Specifically, a linked binary vector, pRIBAC LacSG-tetPF, was introduced into BY-2, and selection using the Hyg resistance gene mounted on the LacSG side as a marker was first performed. Subsequently, among the obtained transformants, strains in which the expression of the EYFP gene mounted on the tetPF side were selected by the genomic PCR and observation of EYFP fluorescence. As a result, multiple expected transformants, i.e., strains expressing the Hyg resistance gene on the LacSG side and repressing the EYFP gene on the tetPF side could be acquired (FIG. 44).

The present invention has a high advantage in that commercialization is possible by only production of few genetically modified strains in a short period of time, and it is expected that this technology will be widely used in the field of plant biotechnology in the future. In addition, this generic technology enables commercialization by production of few genetically modified strains not only in plant species whose genetic modification is easy but also in plant species whose hurdle for commercialization is conventionally high because of difficulty in genetic modification.

1. A plant recombinant gene expression regulatory platform DNA sequence comprising:
(1) an artificial alphoid DNA sequence having a hierarchical repetitive structure of an alphoid DNA that forms a centromere of a human chromosome and having a nucleotide sequence a part of which is replaced by a binding site of a gene expression regulator (inducer); and
(2) a multigene (a plurality of genes) expression cassette sequence, wherein
the artificial alphoid DNA sequence is linked to upstream (5′ side) and downstream (3′ side) of the cassette sequence.

2. The platform DNA sequence according to claim 1, wherein the alphoid DNA is an α21-1 sequence of human chromosome 21.

3. The platform DNA sequence according to claim 1, wherein the artificial alphoid DNA sequence has a 4 to 16-time repeated structure of a higher ordered repeat unit consisting of 11-mer of a repeat unit of 171 bp, and has a length of 7.5 to 30 kb.

4. The platform DNA sequence according to claim 3, comprising a nucleotide sequence including a binding site of the gene expression regulator in at least a part of the repeat units not containing a CENP-B box sequence.

5. The platform DNA sequence according to claim 4, wherein
the higher ordered repeat unit consisting of the 11-mer of the repeat unit constituting the artificial alphoid DNA sequence is a following DNA sequence:
a DNA sequence (1) consisting of the nucleotide sequence of SEQ ID NO: 1 (including tetO) or SEQ ID NO: 2 (including LacO);
a DNA sequence (2) that hybridizes with a DNA sequence consisting of a nucleotide sequence complementary to the nucleotide sequence of the DNA sequence (1) under stringent conditions, the DNA sequence (2) consisting of a nucleotide sequence that includes a binding site of the gene expression regulator (inducer), and having substantially the same plant recombinant gene expression regulatory function as that of the DNA sequence (1); or
a DNA sequence (3) that consists of a nucleotide sequence showing a sequence identity of 80% or more to the whole of the nucleotide sequence of the DNA sequence (1) and including a binding site of the gene expression regulator (inducer), the DNA sequence (3) having substantially the same plant recombinant gene expression regulatory function as that of the DNA sequence (1).

6. The platform DNA sequence according to claim 1, wherein the binding site of the gene expression regulator is tetO or LacO.

7. The platform DNA sequence according to claim 1, wherein the cassette sequence includes a recombinant enzyme site.

8. The platform DNA sequence according to claim 1, wherein the cassette sequence includes a marker gene or a reporter gene.

9. The platform DNA sequence according to claim 1, comprising several to ten-and-several genes or a multigene having a length of 5 to 100 kb introduced into the cassette sequence.

10. A plant recombinant gene expression regulatory platform vector comprising the platform DNA sequence according to claim 1.

11. A DNA sequence for expressing the gene expression regulator according to claim 1.

12. A gene expression regulator vector comprising the DNA sequence for expressing according to claim 11.

13. The gene expression regulator vector according to claim 12, wherein an artificial alphoid DNA sequence having a hierarchical repetitive structure of an alphoid DNA that forms a centromere of a human chromosome and having a nucleotide sequence a part of which is replaced by a binding site of a gene expression regulator (inducer) is linked to upstream (5′ side) and downstream (3′ side) of the DNA sequence for expression.

14. A plant gene recombinant fusion vector comprising the platform DNA sequence according to claim 1 and a DNA sequence for expressing the gene expression regulator.

15. A plant gene recombinant vector set consisting of the plant recombinant gene expression regulatory platform vector according to claim 10 and a gene expression regulator vector comprising a DNA sequence for expressing the gene expression regulator.

16. A genetically modified plant body comprising a chromosome into which the plant recombinant gene expression regulatory platform DNA sequence according to claim 1 and a DNA sequence for expressing the gene expression regulator have been introduced.

17. A method for manufacturing a plant body, comprising introducing the fusion vector according to claim 14 into the plant body.

18. The manufacturing method according to claim 17, using an Agrobacterium method.

19. A method for regulating the expression of a recombinant gene in a plant body, the method comprising:
repressing or promoting the binding of the gene expression regulator to a binding site included in the artificial alphoid DNA sequence according to claim 1 to regulate action of the gene expression regulator and to thereby transform a chromatin structure on the artificial alphoid DNA sequence and the multigene (a plurality of genes) expression cassette sequence between the heterochromatin and the open chromatin.

20. The regulating method according to claim 19, wherein the repression or promotion of binding of the gene expression regulator to the binding site included in the artificial alphoid DNA sequence is performed by action of tetracycline.

21. The regulating method according to claim 19, wherein the transformation of the chromatin structure is induced by acetylation, methylation, and deacetylation of histone that are actions of the gene expression regulator.

22. A method for manufacturing a plant body, comprising introducing the plant gene recombinant vector set according to claim 15 into the plant body.

23. The manufacturing method according to claim 22, using an Agrobacterium method.


SEQUENCE LISTING (21 sequences)

SEQ ID NO: 1 - DNA, 1886 bp, Artificial Sequence
  tetO 11 mer
gctagcaataaaaggtagacagcagcattctcagaaatttctttctgatgtctgcattcaactcatagagttgaagattgcctttcatagagcaggtttgaaacactctttctggagtatctggatgtggacatttactagcagcagagctctccctatcagtgatagagactagccataaaaacgagacagaaggattctcagaaacaagtttgtgatgtgtgtactcagctaacagagtggaacctttctttttacagagcagctttgaaactctatttttgtggattctgcaaattgatatttaactagcagcagagctctccctatcagtgatagagactagatacaaaatctagacagaagcattctcacaaacttctttgtgatgtgtgtcctcaactaacagagttgaacctttcttttgatgcagcagtttggaaacactctttttgtagaaactgtaagtggatatttggatagctctaacgatttcgttggaaacgggaatatcatgatctaaaatctagacagaagcactattagaaactacttggtgatatctgcattcaagtcacagagttgaacattcccttactttgagcacgtttgaaacactcttttggaagaatctggaagtggacatttactagcagcagagctctccctatcagtgatagagactagccaataaaagccagacagaagctttctcagaaacttgttcgtgatgtgtgtactcaactaaaagagttgaacctttctattgatagagcagttttgaaacactctttttgtggattctgcaagtggatatttggattgctttgaggatttcgttggaagcgggatcactagctataaaaactagacagcagcattcccagaaatttctttgggatatttccattcaactcatagagatgaacatggcctttcatagagcaggtttgaaacactctttttgtagtttgtggaagtggacatttactagcagcagagctctccctatcagtgatagagactagcccataaaaatagacagaagcattctcagaaacttgttggtgatatgtgtcctcaactaacagagttgaactttgccattgatagagagcagttttgaaacactctttttgtggaatctgcaagtggatatttgactagctgactcagaattcgttggaaacgggatcactagcaaataaaaggtagacagcagcattctcagaaatttctttctgatgactgcattcaactcatagagttgaacattccctttcatagagcaggtttgaaacactctttctggagtatctggatgtggacatttactagcagcagagctctccctatcagtgatagagactagcccataaaaacgagacagaaggattctgagaaacaagcttgtgatgtgtgtactcagctaacagagtggaacctctcttttgatgcagcagtttggaaacactctttttgtagaaactgtaagtggatatttggatagctctaatgatttcgttggaaacgggaatatcatcatctaaaatctagacagaagccctctcagaaactactttgtgatatctgcattcaagtcacagagttgaacattcgctttcttagagcacgttggaaacactctttttgtagtgtctggaagtggacatttactagcagcagagctctccctatcagtgatagagactagcccataaaaactagacagaagcatgctcagaaacttgtttttgatgtgtgtacccagccaaaggagttgaacatttctattgatagagcagttttgaaacactctttttgtggaaaatgcaggtggatatttggatagcttggaggatttcgttggaagcgggatcactagt

SEQ ID NO: 2 - DNA, 1886 bp, Artificial Sequence
  LacO 11mer
gctagcaataaaaggtagacagcagcattctcagaaatttctttctgatgtctgcattcaactcatagagttgaagattgcctttcatagagcaggtttgaaacactctttctggagtatctggatgtggacatttggaccacatgtggaattgtgagcggataacaatttgtggccataaaaacgagacagaaggattctcagaaacaagtttgtgatgtgtgtactcagctaacagagtggaacctttctttttacagagcagctttgaaactctatttttgtggattctgcaaattgatatttaggaccacatgtggaattgtgagcggataacaatttgtggatacaaaatctagacagaagcattctcacaaacttctttgtgatgtgtgtcctcaactaacagagttgaacctttcttttgatgcagcagtttggaaacactctttttgtagaaactgtaagtggatatttggatagctctaacgatttcgttggaaacgggaatatcatgatctaaaatctagacagaagcactattagaaactacttggtgatatctgcattcaagtcacagagttgaacattcccttactttgagcacgtttgaaacactcttttggaagaatctggaagtggacatttggaccacatgtggaattgtgagcggataacaatttgtggccaataaaagccagacagaagctttctcagaaacttgttcgtgatgtgtgtactcaactaaaagagttgaacctttctattgatagagcagttttgaaacactctttttgtggattctgcaagtggatatttggattgctttgaggatttcgttggaagcgggatcactagctataaaaactagacagcagcattcccagaaatttctttgggatatttccattcaactcatagagatgaacatggcctttcatagagcaggtttgaaacactctttttgtagtttgtggaagtggacatttcgaccacatgtggaattgtgagcggataacaatttgtggcccataaaaatagacagaagcattctcagaaacttgttggtgatatgtgtcctcaactaacagagttgaactttgccattgatagagagcagttttgaaacactctttttgtggaatctgcaagtggatatttgactagctgactcagaattcgttggaaacgggatcactagcaaataaaaggtagacagcagcattctcagaaatttctttctgatgactgcattcaactcatagagttgaacattccctttcatagagcaggtttgaaacactctttctggagtatctggatgtggacatttggaccacatgtggaattgtgagcggataacaatttgtggcccataaaaacgagacagaaggattctgagaaacaagcttgtgatgtgtgtactcagctaacagagtggaacctctcttttgatgcagcagtttggaaacactctttttgtagaaactgtaagtggatatttggatagctctaatgatttcgttggaaacgggaatatcatcatctaaaatctagacagaagccctctcagaaactactttgtgatatctgcattcaagtcacagagttgaacattcgctttcttagagcacgttggaaacactctttttgtagtgtctggaagtggacatttggaccacatgtggaattgtgagcggataacaatttgtggcccataaaaacgagacagaagcatgctcagaaacttgtttttgatgtgtgtacccagccaaaggagttgaacatttctattgatagagcagttttgaaacactctttttgtggaaaatgcaggtggatatttggatagcttggaggatttcgttggaagcgggatcactagt

SEQ ID NO: 3 - DNA, 5403 bp, Artificial Sequence
  pBS KMNeo
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgataaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 4 - DNA, 10692 bp, Artificial Sequence
  pRIBAC
tttaagcttaaagcatgcaaagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtcacatacaaatggacgaacggataaaccttttcacgcccttttaaatatccgattattctaataaacgctcttttctcttaggtttacccgccaatatatcctgtcatttggatccatagctagctttagtacttttactagtggaaccaaagcggccgctttgtttacaccacaatatatcctgccacaagctagctttctgagccgccgattttcctcctcgagttggatgaactcgccgagttcatcgtcaactgaaacagacacggccggattctgtgagacaggttgaaccgcagctctcttccattgataataggtctgaacggaaatacccacgatcttaacggcgtccttcaaggttgcgccgccagcgacctgagcttcgatttgaccgatcttctccagtttttctcggttgctgaggccgcgggttttcggcttcacggatttgaacgatcccgtgcgggctgtttcggctggtgctttctttgctcttctacctctaggagcagccggctcaacttcggcagcagcagtaccgtccggcggattctggatctcttcgtcagccattaatcgtcctctgtgtgggttattgctttgtctgccagctcgatccaagagtcaacgtttgtgcctagggcagtaaataggcagtgctccgcgactacatgcctcggccggcaaaataccgccgcatgtagagcaggctctccttcacgatcaacgatcggcatggggccttcgtgcttgttgagtaatgttatcgctcccatcagagcacgcttggtactccgggaatcggatggtctgtcgatcatccaaaaaacgctcatgttttcaacctattaggtctgtggtcagctgaccacagaccatcctgctccatactcgctaattctagccaaaccgcaacgtcccctgcccgctagccttcaagagcgccattatcatcgggccaagtgaaaacttcccgagttcgctccgccgtgtcagatctcggagatagcccccaggcgaattgatgaagttcgctcgctccaaaatgcacgccatcgctgctgccgcattctccggtcccattgcctcacacgcgtcttggtaagccgacgggctgacccccagcatagaccgaaccaccaccgcagccgacatgaggtcacgccagctagcaaccgcaccgctcggcccataattgccaatggtcgggcacgctttcaggatcatcccgagggggaacgcttttatcggctcgctccttgcccggtctatttcactcggcttagcgccctgctccttttcagagcgaggttcaagttcattaacggattcgggttttgagttctgtatgtgctgctcgctctgggcagcattggtgctattattttctgaattgtctctaatttccaaccggttgattatctcttcctggagcatccacatctcttcgagaattgactctacatcagcaagcgtcggggcgcgtgggattctacccacaagttccacatagacttcctcgacagcttgccagtcgccctccgctccctcttccatagctgccgtaattagcttccgaacgtcccgtcggcaaatcgtcagactttctttggccatcctgaatgctgctcgatcggccatcacctgctgtgccatcatcgctagctcttcggaccgcgcgagaagcggagacaaatcgaagccaaacgcgcgctcgatctgaccagcgccatccttacgagcgtaacgctttccgttggcgctatccttccggacgatcaagcctgactccacgagcatggcgatgtgcctacgcaaagtcgcgccagccatcccatgcgcccgaagggcaagctgagcattcgacgggaagacgatcagctgtgcctcctgacgcaactccgtttccgggtgaaagctcaatagcgcatcaaggacggcaagactgttggactggattccaagtagttccatggccgcggacgcgtccctaaagaccttccacttgtccgctgtcttgccttgtttgatatcggccagcgccgtctggcgccgcacaagcgcaagcgtcattggccgccgcccgaatggcgtcgttacacttcctgtctgcatcatctttcacctttcagcaggcaaaggaaatcagctcaccaaaacggcgctaaaaactcttgacgaggattcgaggaaatgcgattctgttcgcgctagagagacagaagggcttccgcgacggcgacgttgagggggctcttttcttttgcggtttactctccccgtttccgttggttctcagcgtggtacgcttgatacagcgctggcacatgatcgagcacgaaggtcgcaaaatcgggcgtcgccttcctgtcaatcgtgatttccagtttggccttgctctgcgtcacctgtgcaattctggtgccgtctggggtggccatgacctcgggaagtccacgcgcaacccgactgggcttcagactagcgatcaccgccttgaatcgttctgccgatggcagcgcttgaacttcctccgacatagcatatttagccacgtcggccggtgaagaaactttctcaatcagctcggcaagttgttgccaactcggccgtccaacaccaggagcggcaccaatagcatcggtcagttcagaggggagggcgtcaacgagcagaagcatcttggacaaattgctcttgtcgatcgacatcgcggcgatgacaatctctcgagaaaactgcctgttcaggcgatgtgcgaagcgcgccttttcgatgaaggtaagatcttcgcgcacattgttttcctgaccctgtgctacgaccacttgctcgtccgtcagttcgcgaacgaccgccctgaccggaagtccgagttctgaaacggcgcgtagccggcggtggccgaaggcaacctgatatcggcccggctggctcggatgcggtcgcacaaggattgggacttgctgtccttgttcccggatcgaagtaaggagcccgtcaatgtcccctcgcatacgatcctgcacgaaagacggttctattgacgaggcatccaactctatcactgcctgaccttcagcgagacgccgctcgatctcttcggcacggctaagacgatcgttttgctctcgcagtgcgttaccaatgttcgctgtgagcttcgttgccggatcgcgctccttccttgttacgccgaggagcggcatggagcggttctttgccgtcctattgtcggcgggcgacgtctcaggggcgtcagttgagacgccaaggatgtgcttccggctcatgtgggcctaccccatgcttttttgatcagtgtttcgatctcgtcgttgacggcgttcatcgcctccaaggctcgatcataggtcgagcgcgtgaacaggccacgctccacttcgaatagagtctggtttgtcaggccagcgtccgaaaccgcggtggttttaagcatcggaaaattgaggacattttcgccaaaaatcgaccgcagataacctaccatttggttctgtggtccgtcgctcggttcgaaacgggttatcagatagcgcatccaattaaacttgaacttggcgccagcattctcgatttcacgcaaaaggttcgatgtcattgccagaaactggttcatcgacatcacatccagcatctgcggatggaccgtgacaagaatggacgtcgccgcagtcaatgcggatagcgtgagatacccaagctggggagggcagtcgatgaccacgacgtcatagttatccgcgatatcttcaattacttggctgatgcgaccataaaagagcgtgtcgccctctttgcggttcatcagcgcgcgtggcgtatcgtgttcaaactccatcagctcaaggttaccaggaatcaggtggaggtcgggaatgtaagtccctcggacgactcgttcgattgccacctgctcatcatcataccttatagcgccgtagagcgtttcgttcgggccaacgtccgtctccggttggctcccaaagagtgcagaaaggctcgcttgaggatcgagatcaatggccaagactcgatatccgcgcatagcgaggtactgcgccagatgcgcggcggtggtggtcttacccgacccacctttgaaattcatcacagagataacctgaagctgctcgccgcctcgacgatgtggcaggtagcgccggttcccgcggccgacctgatccatatacttccgaatcacatggatatcttcaattgagaacattcgcctgccacctgggctcatgctaacattcaactctggcatctcagacgcggtctgccgtaaatatgactcgccaacgccgagcagcttggacgcctccgatggcccgaatgttcgaatacccttctcggaatgcggcgggaaaaccttaagatgatgtgcttgaagttggctcgagagggcatcggcatgacgctccatcaaggccgtcaaccctacaactacaggcgctgcttttaggacagacttcgccatctcaaacccattccttgccagtggcgatatttttcgcgaaactggaaaagttccgccgctggcaattagcgccgattctgctgtttgggcaagagcttttaggttaacagaaggttaacgccctcaggtcgaaaaactccacccaactgttatttgtatttatttccaatgccttagagagattgccatttgaatatgttcatgtattgttttagtgataatcctacaatcgtaacccaaaaagaggtcgccctctgcgcgccgtcgtccaatataggcgaagtcacccttgcgactcaggcggattctaccttgtaggatctttgaattcaaagagctcagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaagctagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccct

SEQ ID NO: 5 - DNA, 18489 bp, Artificial Sequence
  pRIBAC MGC Alone
tttaagcttaaagcatgcaaagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtcacatacaaatggacgaacggataaaccttttcacgcccttttaaatatccgattattctaataaacgctcttttctcttaggtttacccgccaatatatcctgtcatttggatccatagctagctttagtacttttactagcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaggatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcaactagcaaatatttcttgtcaaaaatgctccactgacgttccataaattcccctcggtatccaattagagtctcatattcactctcaatccaaataatctgcaccggatctggatcgtttcgcactagctaccgttcgtataggatactttatacgaaggtataggatccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactacaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaatttgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttctagaataacttcgtataatgtatgctatacgaacggtactagccgtgattctgagaagtgtctttctcggaaattgaaacactgatagtttaaactgaaggcgggaaacgacaatctgatcctggcgaaagggggatgtgctgcaaggcgattaagttgggtaacgccagggttttcccagtcacgacgttgtaaaacgacggccagtgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatactagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagatatcgttaacactcgacggaggagctggtggtgcaggaggttagactagctcgacctcgacaattctgaatcaacaactctcctggcgcaccatcgtcggctacagcctcgggaattgctaccgagctcttatgaagatgaagatgaaatatttggtgtgtcaaataaaaaactagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtaccttcaatttccgagaatgacagttctcagaatcacgactagctgacggtccgataagtgtatttatcggacatgataacactgatagtttaaactgaaggcgggaaacgacaatctgatcctggcgaaagggggatgtgctgcaaggcgattaagttgggtaacgccagggttttcccagtcacgacgttgtaaaacgacggccagtgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatactagcatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccgggatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaactagctcgacctcgacaattctgaatcaacaactctcctggcgcaccatcgtcggctacagcctcgggaattgctaccgagctcttatgaagatgaagatgaaatatttggtgtgtcaaataaaaaactagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctatcatgtccgataattacagttatcggaccgtcaactagtaaagcggccgctttgtttacaccacaatatatcctgccacaagctagctttctgagccgccgattttcctcctcgagttggatgaactcgccgagttcatcgtcaactgaaacagacacggccggattctgtgagacaggttgaaccgcagctctcttccattgataataggtctgaacggaaatacccacgatcttaacggcgtccttcaaggttgcgccgccagcgacctgagcttcgatttgaccgatcttctccagtttttctcggttgctgaggccgcgggttttcggcttcacggatttgaacgatcccgtgcgggctgtttcggctggtgctttctttgctcttctacctctaggagcagccggctcaacttcggcagcagcagtaccgtccggcggattctggatctcttcgtcagccattaatcgtcctctgtgtgggttattgctttgtctgccagctcgatccaagagtcaacgtttgtgcctagggcagtaaataggcagtgctccgcgactacatgcctcggccggcaaaataccgccgcatgtagagcaggctctccttcacgatcaacgatcggcatggggccttcgtgcttgttgagtaatgttatcgctcccatcagagcacgcttggtactccgggaatcggatggtctgtcgatcatccaaaaaacgctcatgttttcaacctattaggtctgtggtcagctgaccacagaccatcctgctccatactcgctaattctagccaaaccgcaacgtcccctgcccgctagccttcaagagcgccattatcatcgggccaagtgaaaacttcccgagttcgctccgccgtgtcagatctcggagatagcccccaggcgaattgatgaagttcgctcgctccaaaatgcacgccatcgctgctgccgcattctccggtcccattgcctcacacgcgtcttggtaagccgacgggctgacccccagcatagaccgaaccaccaccgcagccgacatgaggtcacgccagctagcaaccgcaccgctcggcccataattgccaatggtcgggcacgctttcaggatcatcccgagggggaacgcttttatcggctcgctccttgcccggtctatttcactcggcttagcgccctgctccttttcagagcgaggttcaagttcattaacggattcgggttttgagttctgtatgtgctgctcgctctgggcagcattggtgctattattttctgaattgtctctaatttccaaccggttgattatctcttcctggagcatccacatctcttcgagaattgactctacatcagcaagcgtcggggcgcgtgggattctacccacaagttccacatagacttcctcgacagcttgccagtcgccctccgctccctcttccatagctgccgtaattagcttccgaacgtcccgtcggcaaatcgtcagactttctttggccatcctgaatgctgctcgatcggccatcacctgctgtgccatcatcgctagctcttcggaccgcgcgagaagcggagacaaatcgaagccaaacgcgcgctcgatctgaccagcgccatccttacgagcgtaacgctttccgttggcgctatccttccggacgatcaagcctgactccacgagcatggcgatgtgcctacgcaaagtcgcgccagccatcccatgcgcccgaagggcaagctgagcattcgacgggaagacgatcagctgtgcctcctgacgcaactccgtttccgggtgaaagctcaatagcgcatcaaggacggcaagactgttggactggattccaagtagttccatggccgcggacgcgtccctaaagaccttccacttgtccgctgtcttgccttgtttgatatcggccagcgccgtctggcgccgcacaagcgcaagcgtcattggccgccgcccgaatggcgtcgttacacttcctgtctgcatcatctttcacctttcagcaggcaaaggaaatcagctcaccaaaacggcgctaaaaactcttgacgaggattcgaggaaatgcgattctgttcgcgctagagagacagaagggcttccgcgacggcgacgttgagggggctcttttcttttgcggtttactctccccgtttccgttggttctcagcgtggtacgcttgatacagcgctggcacatgatcgagcacgaaggtcgcaaaatcgggcgtcgccttcctgtcaatcgtgatttccagtttggccttgctctgcgtcacctgtgcaattctggtgccgtctggggtggccatgacctcgggaagtccacgcgcaacccgactgggcttcagactagcgatcaccgccttgaatcgttctgccgatggcagcgcttgaacttcctccgacatagcatatttagccacgtcggccggtgaagaaactttctcaatcagctcggcaagttgttgccaactcggccgtccaacaccaggagcggcaccaatagcatcggtcagttcagaggggagggcgtcaacgagcagaagcatcttggacaaattgctcttgtcgatcgacatcgcggcgatgacaatctctcgagaaaactgcctgttcaggcgatgtgcgaagcgcgccttttcgatgaaggtaagatcttcgcgcacattgttttcctgaccctgtgctacgaccacttgctcgtccgtcagttcgcgaacgaccgccctgaccggaagtccgagttctgaaacggcgcgtagccggcggtggccgaaggcaacctgatatcggcccggctggctcggatgcggtcgcacaaggattgggacttgctgtccttgttcccggatcgaagtaaggagcccgtcaatgtcccctcgcatacgatcctgcacgaaagacggttctattgacgaggcatccaactctatcactgcctgaccttcagcgagacgccgctcgatctcttcggcacggctaagacgatcgttttgctctcgcagtgcgttaccaatgttcgctgtgagcttcgttgccggatcgcgctccttccttgttacgccgaggagcggcatggagcggttctttgccgtcctattgtcggcgggcgacgtctcaggggcgtcagttgagacgccaaggatgtgcttccggctcatgtgggcctaccccatgcttttttgatcagtgtttcgatctcgtcgttgacggcgttcatcgcctccaaggctcgatcataggtcgagcgcgtgaacaggccacgctccacttcgaatagagtctggtttgtcaggccagcgtccgaaaccgcggtggttttaagcatcggaaaattgaggacattttcgccaaaaatcgaccgcagataacctaccatttggttctgtggtccgtcgctcggttcgaaacgggttatcagatagcgcatccaattaaacttgaacttggcgccagcattctcgatttcacgcaaaaggttcgatgtcattgccagaaactggttcatcgacatcacatccagcatctgcggatggaccgtgacaagaatggacgtcgccgcagtcaatgcggatagcgtgagatacccaagctggggagggcagtcgatgaccacgacgtcatagttatccgcgatatcttcaattacttggctgatgcgaccataaaagagcgtgtcgccctctttgcggttcatcagcgcgcgtggcgtatcgtgttcaaactccatcagctcaaggttaccaggaatcaggtggaggtcgggaatgtaagtccctcggacgactcgttcgattgccacctgctcatcatcataccttatagcgccgtagagcgtttcgttcgggccaacgtccgtctccggttggctcccaaagagtgcagaaaggctcgcttgaggatcgagatcaatggccaagactcgatatccgcgcatagcgaggtactgcgccagatgcgcggcggtggtggtcttacccgacccacctttgaaattcatcacagagataacctgaagctgctcgccgcctcgacgatgtggcaggtagcgccggttcccgcggccgacctgatccatatacttccgaatcacatggatatcttcaattgagaacattcgcctgccacctgggctcatgctaacattcaactctggcatctcagacgcggtctgccgtaaatatgactcgccaacgccgagcagcttggacgcctccgatggcccgaatgttcgaatacccttctcggaatgcggcgggaaaaccttaagatgatgtgcttgaagttggctcgagagggcatcggcatgacgctccatcaaggccgtcaaccctacaactacaggcgctgcttttaggacagacttcgccatctcaaacccattccttgccagtggcgatatttttcgcgaaactggaaaagttccgccgctggcaattagcgccgattctgctgtttgggcaagagcttttaggttaacagaaggttaacgccctcaggtcgaaaaactccacccaactgttatttgtatttatttccaatgccttagagagattgccatttgaatatgttcatgtattgttttagtgataatcctacaatcgtaacccaaaaagaggtcgccctctgcgcgccgtcgtccaatataggcgaagtcacccttgcgactcaggcggattctaccttgtaggatctttgaattcaaagagctcagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaagctagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccct

SEQ ID NO: 6 - DNA, 22760 bp, Artificial Sequence
  pRIBAC SG Alone
tttaagcttaaagcatgcaaagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtcacatacaaatggacgaacggataaaccttttcacgcccttttaaatatccgattattctaataaacgctcttttctcttaggtttacccgccaatatatcctgtcatttggatccaaagctagcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaggatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcaactagcaaatatttcttgtcaaaaatgctccactgacgttccataaattcccctcggtatccaattagagtctcatattcactctcaatccaaataatctgcaccggatctggatcgtttcgcactagcatggatagatccggaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagactagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaatttgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttactagcaacactgatagtttaaactgaaggcgggaaacgacaatctgatcctggcgaaagggggatgtgctgcaaggcgattaagttgggtaacgccagggttttcccagtcacgacgttgtaaaacgacggccagtgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatactagcatgtctagagtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgaggcggaaaaaagaagagaaagggaggaggtgcaggaggtgctactagcgcaccaacggagaggcagcttcgatataaggaaaaagtggctgaactcaggaagaaaagaaattctggactgagcaaagaacagaaagagaaatatatggaacacagacagacctatgggaacacacgggaacctcttttagaaaacctgacaagcgagtatgacttggatcttttccgaagagcacaagcccgggcttcagaggatttggagaagttaaggctgcaaggccaaatcacagagggaagcaacatgattaaaacaattgcttttggccgctatgagcttgatacctggtatcattctccatatcctgaagaatatgcacggctgggacgtctctatatgtgtgaattctgtttaaaatatatgaagagccaaacgatactccgccggcacatggccaaatgtgtgtggaaacacccacctggtgatgagatatatcgcaaaggttcaatctctgtgtttgaagtggatggcaagaaaaacaagatctactgccaaaacctgtgcctgttggccaaactttttctggaccacaagacattatattatgatgtggagcccttcctgttctatgttatgacagaggcggacaacactggctgtcacctgattggatatttttctaaggaaaagaattcattcctcaactacaacgtctcctgtatccttactatgcctcagtacatgagacagggctatggcaagatgcttattgatttcagttatttgctttccaaagtcgaagaaaaagttggctccccagaacgtccactctcagatctggggcttataagctatcgcagttactggaaagaagtacttctccgctacctgcataattttcaaggcaaagagatttctatcaaagaaatcagtcaggagacggctgtgaatcctgtggacattgtcagcactctgcaagcccttcagatgctcaaatactggaagggaaaacacctagttttaaagagacaggacctgattgatgagtggatagccaaagaggccaaaaggtccaactccaataaaaccatggatcccagctgcttaaaatggacccctcccaagggcacttaaactagctcgacctcgacaattctgaatcaacaactctcctggcgcaccatcgtcggctacagcctcgggaattgctaccgagctcttatgaagatgaagatgaaatatttggtgtgtcaaataaaaaactagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctactagcaacactgatagtttaaactgaaggcgggaaacgacaatctgatcctggcgaaagggggatgtgctgcaaggcgattaagttgggtaacgccagggttttcccagtcacgacgttgtaaaacgacggccagtgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatactagcatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacccaaaaaagaagagaaagaggcctccgctaggaggaggtgctggtggcggaactagcgcaccaacggagaggcagcttcgatataaggaaaaagtggctgaactcaggaagaaaagaaattctggactgagcaaagaacagaaagagaaatatatggaacacagacagacctatgggaacacacgggaacctcttttagaaaacctgacaagcgagtatgacttggatcttttccgaagagcacaagcccgggcttcagaggatttggagaagttaaggctgcaaggccaaatcacagagggaagcaacatgattaaaacaattgcttttggccgctatgagcttgatacctggtatcattctccatatcctgaagaatatgcacggctgggacgtctctatatgtgtgaattctgtttaaaatatatgaagagccaaacgatactccgccggcacatggccaaatgtgtgtggaaacacccacctggtgatgagatatatcgcaaaggttcaatctctgtgtttgaagtggatggcaagaaaaacaagatctactgccaaaacctgtgcctgttggccaaactttttctggaccacaagacattatattatgatgtggagcccttcctgttctatgttatgacagaggcggacaacactggctgtcacctgattggatatttttctaaggaaaagaattcattcctcaactacaacgtctcctgtatccttactatgcctcagtacatgagacagggctatggcaagatgcttattgatttcagttatttgctttccaaagtcgaagaaaaagttggctccccagaacgtccactctcagatctggggcttataagctatcgcagttactggaaagaagtacttctccgctacctgcataattttcaaggcaaagagatttctatcaaagaaatcagtcaggagacggctgtgaatcctgtggacattgtcagcactctgcaagcccttcagatgctcaaatactggaagggaaaacacctagttttaaagagacaggacctgattgatgagtggatagccaaagaggccaaaaggtccaactccaataaaaccatggatcccagctgcttaaaatggacccctcccaagggcacttaaactagctcgacctcgacaattctgaatcaacaactctcctggcgcaccatcgtcggctacagcctcgggaattgctaccgagctcttatgaagatgaagatgaaatatttggtgtgtcaaataaaaaactagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctactagcaacactgatagtttaaactgaaggcgggaaacgacaatctgatcctggcgaaagggggatgtgctgcaaggcgattaagttgggtaacgccagggttttcccagtcacgacgttgtaaaacgacggccagtgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatactagcatgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctagccccccccagtttgaacaagcagaggcgcaactacgctgtctatgcgatgcagggttttcggtcgaggaggctcttttcattctgcaatctataagccattttagcttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagaggcctccgctaggaggaggtgctggtggcggaactagctaccgtgttggtgagggcatcaccctcaaccaggtggctgtgggctgcgagtgccaggactgtctgtgggcacccactggaggctgctgcccgggggcgtcactgcacaagtttgcctacaatgaccagggccaggtgcggcttcgagccgggctgcccatctacgagtgcaactcccgctgccgctgcggctatgactgcccaaatcgtgtggtacagaagggtatccgatatgacctctgcatcttccgcacggatgatgggcgtggctggggcgtccgcaccctggagaagattcgcaagaacagcttcgtcatggagtacgtgggagagatcattacctcagaggaggcagagcggcggggccagatctacgaccgtcagggcgccacctacctctttgacctggactacgtggaggacgtgtacaccgtggatgccgcctactatggcaacatctcccactttgtcaaccacagttgtgaccccaacctgcaggtgtacaacgtcttcatagacaaccttgacgagcggctgccccgcatcgctttctttgccacaagaaccatccgggcaggcgaggagctcacctttgattacaacatgcaagtggaccccgtggacatggagagcacccgcatggactccaactttggcctggctgggctccctggctcccctaagaagcgggtccgtattgaatgcaagtgtgggactgagtcctgccgcaaatacctcttctagactagctcgacctcgacaattctgaatcaacaactctcctggcgcaccatcgtcggctacagcctcgggaattgctaccgagctcttatgaagatgaagatgaaatatttggtgtgtcaaataaaaaactagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctactagtaaagcggccgctttgtttacaccacaatatatcctgccacaagctagctttctgagccgccgattttcctcctcgagttggatgaactcgccgagttcatcgtcaactgaaacagacacggccggattctgtgagacaggttgaaccgcagctctcttccattgataataggtctgaacggaaatacccacgatcttaacggcgtccttcaaggttgcgccgccagcgacctgagcttcgatttgaccgatcttctccagtttttctcggttgctgaggccgcgggttttcggcttcacggatttgaacgatcccgtgcgggctgtttcggctggtgctttctttgctcttctacctctaggagcagccggctcaacttcggcagcagcagtaccgtccggcggattctggatctcttcgtcagccattaatcgtcctctgtgtgggttattgctttgtctgccagctcgatccaagagtcaacgtttgtgcctagggcagtaaataggcagtgctccgcgactacatgcctcggccggcaaaataccgccgcatgtagagcaggctctccttcacgatcaacgatcggcatggggccttcgtgcttgttgagtaatgttatcgctcccatcagagcacgcttggtactccgggaatcggatggtctgtcgatcatccaaaaaacgctcatgttttcaacctattaggtctgtggtcagctgaccacagaccatcctgctccatactcgctaattctagccaaaccgcaacgtcccctgcccgctagccttcaagagcgccattatcatcgggccaagtgaaaacttcccgagttcgctccgccgtgtcagatctcggagatagcccccaggcgaattgatgaagttcgctcgctccaaaatgcacgccatcgctgctgccgcattctccggtcccattgcctcacacgcgtcttggtaagccgacgggctgacccccagcatagaccgaaccaccaccgcagccgacatgaggtcacgccagctagcaaccgcaccgctcggcccataattgccaatggtcgggcacgctttcaggatcatcccgagggggaacgcttttatcggctcgctccttgcccggtctatttcactcggcttagcgccctgctccttttcagagcgaggttcaagttcattaacggattcgggttttgagttctgtatgtgctgctcgctctgggcagcattggtgctattattttctgaattgtctctaatttccaaccggttgattatctcttcctggagcatccacatctcttcgagaattgactctacatcagcaagcgtcggggcgcgtgggattctacccacaagttccacatagacttcctcgacagcttgccagtcgccctccgctccctcttccatagctgccgtaattagcttccgaacgtcccgtcggcaaatcgtcagactttctttggccatcctgaatgctgctcgatcggccatcacctgctgtgccatcatcgctagctcttcggaccgcgcgagaagcggagacaaatcgaagccaaacgcgcgctcgatctgaccagcgccatccttacgagcgtaacgctttccgttggcgctatccttccggacgatcaagcctgactccacgagcatggcgatgtgcctacgcaaagtcgcgccagccatcccatgcgcccgaagggcaagctgagcattcgacgggaagacgatcagctgtgcctcctgacgcaactccgtttccgggtgaaagctcaatagcgcatcaaggacggcaagactgttggactggattccaagtagttccatggccgcggacgcgtccctaaagaccttccacttgtccgctgtcttgccttgtttgatatcggccagcgccgtctggcgccgcacaagcgcaagcgtcattggccgccgcccgaatggcgtcgttacacttcctgtctgcatcatctttcacctttcagcaggcaaaggaaatcagctcaccaaaacggcgctaaaaactcttgacgaggattcgaggaaatgcgattctgttcgcgctagagagacagaagggcttccgcgacggcgacgttgagggggctcttttcttttgcggtttactctccccgtttccgttggttctcagcgtggtacgcttgatacagcgctggcacatgatcgagcacgaaggtcgcaaaatcgggcgtcgccttcctgtcaatcgtgatttccagtttggccttgctctgcgtcacctgtgcaattctggtgccgtctggggtggccatgacctcgggaagtccacgcgcaacccgactgggcttcagactagcgatcaccgccttgaatcgttctgccgatggcagcgcttgaacttcctccgacatagcatatttagccacgtcggccggtgaagaaactttctcaatcagctcggcaagttgttgccaactcggccgtccaacaccaggagcggcaccaatagcatcggtcagttcagaggggagggcgtcaacgagcagaagcatcttggacaaattgctcttgtcgatcgacatcgcggcgatgacaatctctcgagaaaactgcctgttcaggcgatgtgcgaagcgcgccttttcgatgaaggtaagatcttcgcgcacattgttttcctgaccctgtgctacgaccacttgctcgtccgtcagttcgcgaacgaccgccctgaccggaagtccgagttctgaaacggcgcgtagccggcggtggccgaaggcaacctgatatcggcccggctggctcggatgcggtcgcacaaggattgggacttgctgtccttgttcccggatcgaagtaaggagcccgtcaatgtcccctcgcatacgatcctgcacgaaagacggttctattgacgaggcatccaactctatcactgcctgaccttcagcgagacgccgctcgatctcttcggcacggctaagacgatcgttttgctctcgcagtgcgttaccaatgttcgctgtgagcttcgttgccggatcgcgctccttccttgttacgccgaggagcggcatggagcggttctttgccgtcctattgtcggcgggcgacgtctcaggggcgtcagttgagacgccaaggatgtgcttccggctcatgtgggcctaccccatgcttttttgatcagtgtttcgatctcgtcgttgacggcgttcatcgcctccaaggctcgatcataggtcgagcgcgtgaacaggccacgctccacttcgaatagagtctggtttgtcaggccagcgtccgaaaccgcggtggttttaagcatcggaaaattgaggacattttcgccaaaaatcgaccgcagataacctaccatttggttctgtggtccgtcgctcggttcgaaacgggttatcagatagcgcatccaattaaacttgaacttggcgccagcattctcgatttcacgcaaaaggttcgatgtcattgccagaaactggttcatcgacatcacatccagcatctgcggatggaccgtgacaagaatggacgtcgccgcagtcaatgcggatagcgtgagatacccaagctggggagggcagtcgatgaccacgacgtcatagttatccgcgatatcttcaattacttggctgatgcgaccataaaagagcgtgtcgccctctttgcggttcatcagcgcgcgtggcgtatcgtgttcaaactccatcagctcaaggttaccaggaatcaggtggaggtcgggaatgtaagtccctcggacgactcgttcgattgccacctgctcatcatcataccttatagcgccgtagagcgtttcgttcgggccaacgtccgtctccggttggctcccaaagagtgcagaaaggctcgcttgaggatcgagatcaatggccaagactcgatatccgcgcatagcgaggtactgcgccagatgcgcggcggtggtggtcttacccgacccacctttgaaattcatcacagagataacctgaagctgctcgccgcctcgacgatgtggcaggtagcgccggttcccgcggccgacctgatccatatacttccgaatcacatggatatcttcaattgagaacattcgcctgccacctgggctcatgctaacattcaactctggcatctcagacgcggtctgccgtaaatatgactcgccaacgccgagcagcttggacgcctccgatggcccgaatgttcgaatacccttctcggaatgcggcgggaaaaccttaagatgatgtgcttgaagttggctcgagagggcatcggcatgacgctccatcaaggccgtcaaccctacaactacaggcgctgcttttaggacagacttcgccatctcaaacccattccttgccagtggcgatatttttcgcgaaactggaaaagttccgccgctggcaattagcgccgattctgctgtttgggcaagagcttttaggttaacagaaggttaacgccctcaggtcgaaaaactccacccaactgttatttgtatttatttccaatgccttagagagattgccatttgaatatgttcatgtattgttttagtgataatcctacaatcgtaacccaaaaagaggtcgccctctgcgcgccgtcgtccaatataggcgaagtcacccttgcgactcaggcggattctaccttgtaggatctttgaattcaaagagctcagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaagctagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccct

SEQ ID NO: 7 - DNA, 34 bp, Artificial Sequence
  Site Specific Recombination Sequence LoxP
ataacttcgtatagcatacattatacgaagttat

SEQ ID NO: 8 - DNA, 34 bp, Artificial Sequence
  Site Specific Recombination Sequence Lox71
taccgttcgtatagcatacattatacgaagttat

SEQ ID NO: 9 - DNA, 34 bp, Artificial Sequence
  Site Specific Recombination Sequence Lox71 2272
taccgttcgtataggatactttatacgaagttat

SEQ ID NO: 10 - DNA, 34 bp, Artificial Sequence
  Site Specific Recombination Sequence Lox66
ataacttcgtatagcatacattatacgaacggta

SEQ ID NO: 11 - DNA, 34 bp, Artificial Sequence
  Site Specific Recombination Sequence Lox66 2272
ataacttcgtataggatactttatacgaacggta

SEQ ID NO: 12 - DNA, 34 bp, Artificial Sequence
  Site Specific Recombination Sequence VLoxP
tcaatttctgagaactgtcattctcggaaattga

SEQ ID NO: 13 - DNA, 34 bp, Artificial Sequence
  Site Specific Recombination Sequence VLox43L
cgtgattctgagaactgtcattctcggaaattga

SEQ ID NO: 14 - DNA, 34 bp, Artificial Sequence
  Site Specific Recombination Sequence VLox43L 2272
cgtgattctgagaagtgtctttctcggaaattga

SEQ ID NO: 15 - DNA, 34 bp, Artificial Sequence
  Site Specific Recombination Sequence VLox43R
tcaatttctgagaactgtcattctcggaatacct

SEQ ID NO: 16 - DNA, 34 bp, Artificial Sequence
  Site Specific Recombination Sequence VLox43R 2272
tcaatttctgagaagtgtctttctcggaatacct

SEQ ID NO: 17 - DNA, 34 bp, Artificial Sequence
  Site Specific Recombination Sequence SLoxP
ctcgtgtccgataactgtaattatcggacatgat

SEQ ID NO: 18 - DNA, 34 bp, Artificial Sequence
  Site Specific Recombination Sequence SLoxV1L
tgacggtccgataactgtaattatcggacatgat

SEQ ID NO: 19 - DNA, 34 bp, Artificial Sequence
  Site Specific Recombination Sequence SLoxV1L 2272
tgacggtccgataagtgtatttatcggacatgat

SEQ ID NO: 20 - DNA, 34 bp, Artificial Sequence
  Site Specific Recombination Sequence SLoxV1R
ctcgtgtccgataactgtaattatcggaccgtca

SEQ ID NO: 21 - DNA, 34 bp, Artificial Sequence
  Site Specific Recombination Sequence SLoxV1R 2272
ctcgtgtccgataagtgtatttatcggaccgtca